(19) United States
(12) Patent Application Publication
(10) Pub. No.: US 2016/0331672 A1
(43) Pub. Date: Nov. 17, 2016

(54) COSMETIC COMPOSITION COMPRISING A POLYMER BEARING A CARBOSILOXANE DENDRIMER UNIT AND EXPANDED POLYMER PARTICLES

(71) Applicant: L'OREAL, Paris (FR)

(72) Inventors: Hélène KHACHIKIAN, Alfortville (FR); Audrey RICARD, La Varenne (FR)

(21) Appl. No.: 15/106,341

(22) PCT Filed: Dec. 11, 2014
(86) PCT No.: PCT/IB2014/066809

§ 371 Date: Jun. 20, 2016

(30) Foreign Application Priority Data
Dec. 20, 2013 (FR) 13 63253

(51) Int. Cl. A61K 8/895; A61Q 1/10; A61K 8/02; A61K 8/06; A61Q 1/02

(52) U.S. Cl. CPC A61K 8/895; A61K 8/064; A61Q 1/02; A61K 8/0279; A61Q 1/10; A61K 2800/544; A61K 2800/43; A61K 2800/594

(57) ABSTRACT

The present invention relates to a cosmetic composition for making up and/or caring for keratin materials, in particular the skin, in the form of a water-in-oil emulsion comprising, in a physiologically acceptable medium, at least one vinyl polymer bearing at least one carbosiloxane dendrimer-based unit, and at least 0.5% by weight of expanded polymer particles relative to the total weight of the composition.

The present invention relates to the field of caring for and/or making up keratin materials, and in particular the skin and mucous membranes. More particularly, the present invention relates to compositions with improved matt-effect performance qualities, especially of the persistence over time of the matt effect.

The term “keratin materials” means the skin, the lips, the eyelashes and the eyebrows, and in particular facial skin or the eyelids, and the eyebrows.

Obtaining a matt effect on the skin is highly desired by users who have combination skin or greasy skin, and also for cosmetic compositions that are intended to be used in hot and humid climates. The highlights caused by an excess of sebum and/or sweat on the surface of the skin are, in effect, generally considered unaesthetic. Shiny skin also generally gives rise to poorer staying power of the makeup, which thus has a tendency to become degraded in the course of the day.

Cosmetic compositions, for instance foundations, are commonly used to give the skin, especially the face, a colour and an aesthetic effect. These makeup products generally contain oils, pigments, fillers and optionally additives such as cosmetic or dermatological active agents.

It is known to those skilled in the art to use fillers in order to obtain a matt effect. These fillers are usually chosen as a function of their noteworthy sebum-absorbing properties and/or their capacity for scattering light. However, their adhesion to the skin is generally poor, above all in the presence of sebum.

Film-forming polymers may then be used to improve the adhesion of these fillers, but also to increase the persistence of the matt effect throughout the day.

Depending on their chemical nature, these polymers may be conveyed in the fatty phase or in the aqueous phase. Examples of these polymers that may be mentioned include silicone resins, polyacrylates, latices, or those described in patent U.S. Pat. No. 6,887,859.

Although the presence of polymers of this type does indeed make it possible to give the cosmetic compositions containing them improved properties of persistence of the matt effect, their presence may, unfortunately, have a detrimental effect on the comfort. Thus, the presence of these polymers may lead to unpleasant sensations and discomfort, either during the application of the composition containing them (difficult spreading, tacky effect, greasy feel, etc.) or in the course of the day (tautness, mask effect, etc.).

More recently, the advantageous properties of vinyl polymers comprising carbosiloxane dendrimer-based units combined with olefin copolymers or silica have been described (see, respectively, EP 1 862 162 and WO 2010/02658).

The need remains for cosmetic compositions that have good cosmetic properties, and which in particular afford an efficient, immediate and long-lasting matt effect.

Thus, the present invention is directed towards proposing cosmetic compositions that have immediate matt effect and further improved persistence of the matt effect, which are pleasant and easy to apply, while at the same time maintaining satisfactory comfort on application, i.e. not causing any sensation of tautness or mask effect throughout the day and/or not giving a greasy or tacky feel during their application.

The invention is also directed towards proposing cosmetic compositions whose makeup homogeneity persistence is also improved.

Moreover, matt foundations are generally anhydrous foundations. These water-free formulations are highly efficient with regard to the immediate matt effect and the matt effect throughout the day, but their powdery finish and the quite greasy application may limit their potential depending on the market.

Thus, the present invention is also directed towards proposing compositions which, besides the abovementioned improved matt-effect performance qualities, afford a more natural and thus less powdery makeup effect with a finer deposit in terms of thickness.

The object of the present invention is to meet these needs.

The present invention thus relates to a cosmetic composition for making up and/or caring for keratin materials, in particular the skin, in the form of a water-in-oil emulsion comprising, in a physiologically acceptable medium, at least one vinyl polymer bearing at least one carbosiloxane dendrimer-based unit, and at least 0.5% by weight of expanded polymer particles relative to the total weight of the composition.

According to a particular mode of the invention, the composition also comprises at least one additional filler, preferably chosen from matt-effect fillers or sebum-absorbing fillers.

The inventors have observed, unexpectedly, that the use in a composition for making up and/or caring for keratin materials, in particular the skin, of a vinyl polymer bearing at least one carbosiloxane dendrimer-based unit in combination with at least 0.5% by weight of expanded polymer particles relative to the total weight of the composition, makes it possible to afford improved performance qualities in terms of persistence of the matt effect, while at the same time maintaining satisfactory comfort on application.

Moreover, and besides the immediate matt-effect performance and the matt-effect performance over time, the nature of the water-in-oil emulsion of a composition according to the invention makes it possible to obtain a more natural, less powdery makeup result and above all a fine film which thus feels less heavy to the user.

Without wishing to be bound by any theory, the inventors have observed that when the content of a vinyl polymer bearing at least one carbosiloxane dendrimer-based unit is less than that of the expanded polymer particles, the persistence of the matt effect is poorer and when the content of expanded polymer particles is less than 0.5% by weight, the cosmetic properties and the matt-effect performance are poorer and/or degraded over time.

A subject of the present invention is also the use in a cosmetic composition, especially of water-in-oil emulsion type, for making up and/or caring for keratin materials, of at least one vinyl polymer bearing at least one carbosiloxane dendrimer-based unit in combination with at least 0.5% by weight of expanded polymer particles relative to the total weight of the composition, to give the said composition improved persistence of the matt effect over time.

The present invention also relates to a process, especially a non-therapeutic process, for making up and/or caring for keratin materials, in particular the skin, comprising at least the step of applying to the said keratin material at least one coat of a composition according to the invention.

The composition of the invention may be a care and/or makeup base composition or a makeup product for keratin materials.

In the case of a makeup base, it is generally applied before a makeup product such as a foundation, to improve the matt effect of the makeup result and the persistence of this matt effect over time.

According to one embodiment, a composition in accordance with the invention may be contained in a container formed from at least one non-thermoplastic material.

According to another embodiment, a composition in accordance with the invention may be contained in a container formed from at least one thermoplastic material.

A cosmetic composition of the invention may be in the form of a care or makeup base, or of a makeup product for keratin materials, in particular for the skin, the lips or the eyebrows, in particular a foundation, a hot-cast foundation product, a lipstick, a body makeup product, a concealer product, an eyeshadow or an eyebrow product.

A care composition according to the invention may in particular be an antisun composition.

It may be fluid or non-fluid, and is advantageously fluid. It may also be in the form of a soft paste.

For the purposes of the present invention, the term “liquid” (or “fluid”) is intended to denote a composition that is capable of flowing under its own weight, at room temperature (20° C.) and at atmospheric pressure (760 mmHg), as opposed to a “solid” composition.

Matt Effect and Persistence of the Matt Effect

The matt effect and the persistence of the matt effect may be measured by means of the protocol described below.

The mattness of a region of skin, for example facial skin, is measured using a polarimetric camera, which is a black and white polarimetric imaging system, with which images are acquired in parallel (P) and crossed (C) polarized light.

By analysing the image resulting from subtraction of the two images (P-C), the brightness is quantified, by measuring the average greyscale of the brightest 5% of pixels corresponding to the bright areas.

More precisely, the measurements are performed on a panel of individuals, for example a sample of 16 women, who are kept in an air-conditioned waiting room (22° C.±2° C.) 15 minutes before the start of the test. They remove their makeup and an image of one of their cheeks is acquired using the polarimetric camera. This image allows measurement of the gloss at T0 before applying makeup. Next, about 100 mg of the cosmetic composition are weighed out on a watch glass, and are applied with the bare fingers onto the half-face on which the measurement at T0 was taken.

After a drying time of 15 minutes, an image of the made-up cheek is acquired using the polarimetric camera. This image allows measurement of the gloss just after applying makeup (Timm). The models then return to the air-conditioned room for 3 hours.

Finally, an image of the made-up cheek after waiting for 3 hours is acquired using the polarimetric camera. This image allows measurement of the gloss after 3 hours of makeup (T3 h).

The results are expressed by calculating the difference (Timm−T0), which measures the effect of the makeup. A negative value means that the makeup reduces the gloss of the skin and that it thus has a matt effect.

The difference (T3 h−Timm) measuring the persistence of this effect is then calculated. The value obtained should be as low as possible, which means that the matt effect of the makeup does not change over time.

Expanded Polymer Particles

A composition according to the invention comprises at least 0.5% by weight of expanded polymer particles relative to the total weight of the composition.

The particles are generally in the form of spheres or beads. It is, however, possible to use particles in the form of fibres or needles.

It is preferable to use particles with a mean particle size of from 1 to 300 microns (m), for example from 5 to 200 μm, preferably from 10 to 100 μm and better still from 15 to 40 μm.

These particles may be made of various inert materials that do not react chemically with the medium; in particular, these particles do not react with the oils, the surfactants, the water or the various other constituents of the composition according to the invention.

The expanded polymer particles are advantageously chosen from particles of thermoplastic materials chosen from polymers or copolymers of acrylonitrile, of vinylidene chloride, of vinyl chloride and/or of an acrylic or styrene monomer, which are optionally expanded, and microporous microspheres.

The acrylic monomer is, for example, a methyl or ethyl acrylate or methacrylate. The styrene monomer is, for example, α-methylstyrene or styrene.

Thus, according to a particular mode, the composition according to the invention comprises hollow particles of expanded acrylonitrile (co)polymer. These particles are thus derived from at least one acrylonitrile polymer or copolymer. They are made of any expanded acrylonitrile polymer or copolymer, which is non-toxic and non-irritant to the skin.

These particles are advantageously spherical in shape. The mass per unit volume of the particles is chosen in the range from 15 kg/m³ to 200 kg/m³, better still from 30 kg/m³ to 120 kg/m³ and even better still from 40 kg/m³ to 80 kg/m³. To obtain this low mass per unit volume, use is advantageously made of expanded polymer or copolymer particles, based on acrylonitrile and preferably on an acrylic or styrene and/or vinylidene chloride monomer.

The internal cavity of the particles contains in principle a gas which may be air, nitrogen or a hydrocarbon such as isobutane or isopentane, preferably isobutane.

Preferably, the particles of the invention have a particle size ranging from 1 μm to 80 μm, better still ranging from 10 μm to 50 μm and better still from 20 μm to 40 μm.

Advantageously, the expanded polymer particles are deformable hollow particles of an expanded copolymer of vinylidene chloride and acrylonitrile or of vinylidene chloride, acrylonitrile and (meth)acrylate or styrene monomer. It is possible, for example, to use a polymer containing 0-60% of units derived from vinylidene chloride, 20-90% of units derived from acrylonitrile and 0-50% of units derived from an acrylic or styrene monomer, the sum of the percentages (by weight) being equal to 100%. The acrylic monomer is, for example, a methyl or ethyl acrylate or methacrylate. The styrene monomer may be styrene or α-methylstyrene.

More preferentially, the expanded polymer particles used in the present invention are hollow particles of an expanded copolymer of vinylidene chloride and acrylonitrile or of vinylidene chloride, acrylonitrile and methyl methacrylate. These particles may be dry or hydrated.

The particles of the invention may be obtained, for example, according to the processes of patents and patent applications EP-56219, EP-348 372, EP-486 080, EP-320 473, EP-112 807 and U.S. Pat. No. 3,615,972.

The expanded polymer particles that may be used in the invention are, for example, microspheres of expanded terpolymer of vinylidene chloride, acrylonitrile and methyl methacrylate, sold under the brand name Expancel by the company Akzo Nobel and in particular under the references 551 DE 12 (particle size D(0.5) of about 12 μm and mass per unit volume of about 40 kg/m³), 551 DE 20 (particle size D(0.5) of about 15 to 25 μm and mass per unit volume of about 60 kg/m³), 551 DE 50 (particle size D(0.5) of about 40 μm), 461 DE 50 and 642 WE 50 of about 50 μm of particle size D(0.5), 551 DE 80 (particle size D(0.5) of about 50 to 80 μm). It is also possible to use particles of this same expanded terpolymer with a particle size D(0.5) of about 18 μm and a mass per unit volume of about 60 to 80 kg/m³ (Expancel EL23) or with a particle size D(0.5) of about 34 μm and a mass per unit volume of about 20 kg/m³. Mention may also be made of the Expancel particles 551 DE 40 d42 (particle size D(0.5) of approximately 30 to 50 μm and mass per unit volume of approximately 42 kg/m³), 551 DE 80 d42 (particle size D(0.5) of approximately 50 to 80 μm and mass per unit volume of approximately 42 kg/m³), 461 DE 20 d70 (particle size D(0.5) of approximately 15 to 25 μm and mass per unit volume of approximately 70 kg/m³), 461 DE 40 d25 (particle size D(0.5) of approximately 35 to 55 μm and mass per unit volume of approximately 25 kg/m³), 461 DE 40 d60 (particle size D(0.5) of approximately 20 to 40 μm and mass per unit volume of approximately 60 kg/m³), 461 DET 40 d25 (particle size D(0.5) of approximately 35 to 55 μm and mass per unit volume of approximately 25 kg/m³), 051 DE 40 d60 (particle size D(0.5) of approximately 20 to 40 μm and mass per unit volume of approximately 60 kg/m³), 091 DE 40 d30 (particle size D(0.5) of approximately 35 to 55 μm and mass per unit volume of approximately 30 kg/m³) or 091 DE 80 d30 (particle size D(0.5) of approximately 60 to 90 μm and mass per unit volume of approximately 30 kg/m³). It is also possible to use particles of a polymer of vinylidene chloride and acrylonitrile or of vinylidene chloride, acrylonitrile and methyl methacrylate in unexpanded form, for instance those sold under the brand name Expancel with the reference 551 DU 10 (particle size D(0.5) of about 10 μm) or 461 DU 15 (particle size D(0.5) of about 15 μm).

According to a particularly preferred embodiment, the expanded polymer particles that may be used in the invention are the particles Expancel 551 DE 40 d42 (particle size D(0.5) of about 30 to 50 μm and mass per unit volume of about 42 kg/m³).

According to a particularly preferred embodiment, a composition according to the invention may comprise from 0.5% to 5% by weight of expanded polymer particles relative to the total weight of the composition, in particular from 0.5% to 2.5% and more particularly from 0.5% to 1.5% by weight relative to the total weight of the composition.

As emerges from the foregoing text, a composition according to the invention uses expanded polymer particles with at least one vinyl polymer bearing at least one carbosiloxane dendrimer derivative.

Advantageously, a composition according to the invention uses a “vinyl polymer(s) bearing at least one carbosiloxane dendrimer derivative/expanded polymer particles” weight ratio of greater than or equal to 6, preferably greater than or equal to 8 and more particularly greater than or equal to 10.

Vinyl Polymer Grafted with a Carbosiloxane Dendrimer

A vinyl polymer that is suitable for preparing a composition according to the invention comprises at least one carbosiloxane dendrimer-based unit.

The vinyl polymer has a backbone and at least one side chain, which comprises a carbosiloxane dendrimer-based unit having a carbosiloxane dendrimer structure.

The term “carbosiloxane dendrimer structure” in the context of the present invention represents a molecular structure with branched groups of high molecular weights, said structure having high regularity in the radial direction starting from the bond to the backbone. Such carbosiloxane dendrimer structures are described in the form of a highly branched siloxane-silylalkylene copolymer in the laid-open Japanese patent application Kokai 9-171 154.

A vinyl polymer according to the invention may contain carbosiloxane dendrimer-based units that may be represented by the following general formula (I):

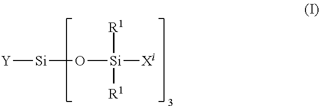

in which:
R¹ represents an aryl group containing from 5 to 10 carbon atoms or an alkyl group containing from 1 to 10 carbon atoms;
Xⁱ represents a silylalkyl group which, when i=1, is represented by formula (II):

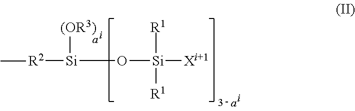

in which:
R¹ is as defined above in formula (I),
R² represents an alkylene radical containing from 2 to 10 carbon atoms,
R³ represents an alkyl group containing from 1 to 10 carbon atoms,
Xⁱ⁺¹ is chosen from: a hydrogen atom, an alkyl group containing from 1 to 10 carbon atoms, an aryl group containing from 5 to 10 carbon atoms and a silylalkyl group defined above of formula (II) with i=i+1,
i is an integer from 1 to 10 which represents the generation of said silylalkyl group, and
aⁱ is an integer from 0 to 3;
Y represents a radical-polymerizable organic group chosen from:
organic groups containing a methacrylic group or an acrylic group, said organic groups being represented by the formulae:

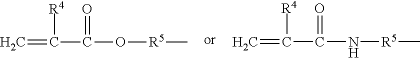

in which:
   R⁴ represents a hydrogen atom or an alkyl group containing from 1 to 10 carbon atoms; and
   R⁵ represents an alkylene group containing from 1 to 10 carbon atoms, such as a methylene group, an ethylene group, a propylene group or a butylene group, methylene and propylene groups being preferred; and
organic groups containing a styryl group of formula:

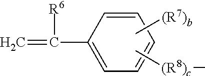

in which:
   R⁶ represents a hydrogen atom or an alkyl group containing from 1 to 10 carbon atoms, such as a methyl group, an ethyl group, a propyl group or a butyl group, the methyl group being preferred;
   R⁷ represents an alkyl group containing from 1 to 10 carbon atoms;
   R⁸ represents an alkylene group containing from 1 to 10 carbon atoms, such as a methylene group, an ethylene group, a propylene group or a butylene group, the ethylene group being preferred;
   b is an integer from 0 to 4; and
   c is 0 or 1, such that, if c is 0, —(R⁸)_{c}— represents a bond.

According to one embodiment, R¹ may represent an aryl group containing from 5 to 10 carbon atoms or an alkyl group containing from 1 to 10 carbon atoms. The alkyl group may preferably be represented by a methyl group, an ethyl group, a propyl group, a butyl group, a pentyl group, an isopropyl group, an isobutyl group, a cyclopentyl group or a cyclohexyl group. The aryl group may preferably be represented by a phenyl group and a naphthyl group. The methyl and phenyl groups are more particularly preferred, and the methyl group is preferred among all.

According to one embodiment, R² represents an alkylene group containing from 2 to 10 carbon atoms, in particular a linear alkylene group, such as an ethylene, propylene, butylene or hexylene group; or a branched alkylene group, such as a methylmethylene, methylethylene, 1-methylpentylene or 1,4-dimethylbutylene group.

Ethylene, methylethylene, hexylene, 1-methylpentylene and 1,4-dimethylbutylene groups are above all preferred.

According to one embodiment, R³ is chosen from methyl, ethyl, propyl, butyl and isopropyl groups.

In formula (II), i indicates the number of generations and thus corresponds to the number of repeats of the silylalkyl group.

For example, when the generation number is equal to 1, the carbosiloxane dendrimer may be represented by the general formula shown below, in which Y, R¹, R² and R³ are as defined above, R¹² represents a hydrogen atom or is identical to R¹; a¹ is identical to aⁱ. Preferably, the total average number of groups OR³ in a molecule is within the range from 0 to 7.

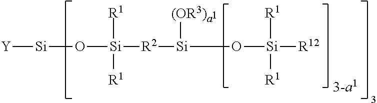

When the generation number is equal to 2, the carbosiloxane dendrimer may be represented by the general formula below, in which Y, R¹, R², R³ and R¹² are the same as defined above; a¹ and a² represent the aⁱ of the indicated generation. Preferably, the total average number of groups OR³ in a molecule is within the range from 0 to 25.

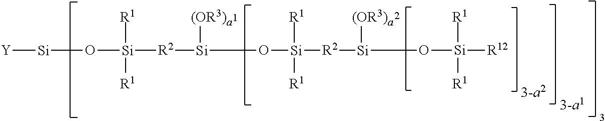

When the generation number is equal to 3, the carbosiloxane dendrimer is represented by the general formula below, in which Y, R¹, R², R³ and R¹² are the same as defined above; a¹, a² and a³ represent the aⁱ of the indicated generation. Preferably, the total average number of groups OR³ in a molecule is within the range from 0 to 79.

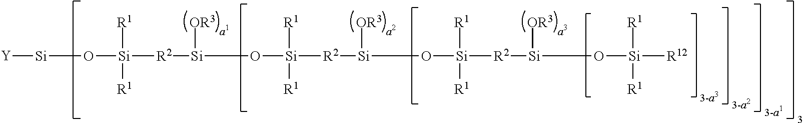

A vinyl polymer bearing at least one carbosiloxane dendrimer-based unit has a molecular side chain containing a carbosiloxane dendrimer structure, and may be derived from the polymerization of:
(A) from 0 to 99.9 parts by weight of a vinyl monomer; and
(B) from 100 to 0.1 parts by weight of a carbosiloxane dendrimer containing a radical-polymerizable organic group, represented by general formula (I) as defined above.

The monomer of vinyl type that is the component (A) in the vinyl polymer bearing at least one carbosiloxane dendrimer-based unit is a monomer of vinyl type that contains a radical-polymerizable vinyl group.

There is no particular limitation as regards such a monomer.

The following are examples of this monomer of vinyl type: methyl methacrylate, ethyl methacrylate, n-propyl methacrylate, isopropyl methacrylate or a methacrylate of an analogous lower alkyl; glycidyl methacrylate; butyl methacrylate, butyl acrylate, n-butyl methacrylate, isobutyl methacrylate, tert-butyl acrylate, tert-butyl methacrylate, n-hexyl methacrylate, cyclohexyl methacrylate, 2-ethylhexyl acrylate, 2-ethylhexyl methacrylate, octyl methacrylate, lauryl methacrylate, stearyl acrylate, stearyl methacrylate or a higher-analogue methacrylate; vinyl acetate, vinyl propionate or a vinyl ester of an analogous lower fatty acid; vinyl caproate, vinyl 2-ethylhexoate, vinyl laurate, vinyl stearate or an ester of a higher fatty acid analogue; styrene, vinyltoluene, benzyl methacrylate, phenoxyethyl methacrylate, vinylpyrrolidone or similar vinylaromatic monomers; methacrylamide, N-methylolmethacrylamide, N-methoxymethylmethacrylamide, isobutoxymethoxymethacrylamide, N,N-dimethylmethacrylamide or similar monomers of vinyl type containing amide groups; hydroxyethyl methacrylate, hydroxypropyl alcohol methacrylate or similar monomers of vinyl type containing hydroxyl groups; acrylic acid, methacrylic acid, itaconic acid, crotonic acid, fumaric acid, maleic acid or similar monomers of vinyl type containing a carboxylic acid group; tetrahydrofurfuryl methacrylate, butoxyethyl methacrylate, ethoxydiethylene glycol methacrylate, polyethylene glycol methacrylate, polypropylene glycol monomethacrylate, hydroxybutyl vinyl ether, cetyl vinyl ether, 2-ethylhexyl vinyl ether or a similar monomer of vinyl type with ether bonds; methacryloxypropyltrimethoxysilane, polydimethylsiloxane containing a methacrylic group on one of its molecular ends, polydimethylsiloxane containing a styryl group on one of its molecular ends, or a similar silicone compound containing unsaturated groups; butadiene; vinyl chloride; vinylidene chloride; methacrylonitrile; dibutyl fumarate; anhydrous maleic acid; anhydrous succinic acid; methacryl glycidyl ether; an organic salt of an amine, an ammonium salt, and an alkali metal salt of methacrylic acid, of itaconic acid, of crotonic acid, of maleic acid or of fumaric acid; a radical-polymerizable unsaturated monomer containing a sulfonic acid group such as a styrenesulfonic acid group; a quaternary ammonium salt derived from methacrylic acid, such as 2-hydroxy-3-methacryloxypropyltrimethylammonium chloride; and a methacrylic acid ester of an alcohol containing a tertiary amine group, such as a methacrylic acid ester of diethylamine.

Multifunctional vinyl monomers may also be used.

The following represent examples of such compounds: trimethylolpropane trimethacrylate, pentaerythrityl trimethacrylate, ethylene glycol dimethacrylate, tetraethylene glycol dimethacrylate, polyethylene glycol dimethacrylate, 1,4-butanediol dimethacrylate, 1,6-hexanediol dimethacrylate, neopentyl glycol dimethacrylate, trimethylolpropanetrioxyethyl methacrylate, tris(2-hydroxyethyl)isocyanurate dimethacrylate, tris(2-hydroxyethyl)isocyanurate trimethacrylate, polydimethylsiloxane capped with styryl groups containing divinylbenzene groups on both ends, or similar silicone compounds containing unsaturated groups.

A carbosiloxane dendrimer, which is the component (B), may be represented by formula (I) as defined above.

The following represent the preferred examples of group Y of formula (I): an acryloxymethyl group, a 3-acryloxypropyl group, a methacryloxymethyl group, a 3-methacryloxypropyl group, a 4-vinylphenyl group, a 3-vinylphenyl group, a 4-(2-propenyl)phenyl group, a 3-(2-propenyl)phenyl group, a 2-(4-vinylphenyl)ethyl group, a 2-(3-vinylphenyl)ethyl group, a vinyl group, an allyl group, a methallyl group and a 5-hexenyl group.

A carbosiloxane dendrimer according to the present invention may be represented by the formulae having the average structures below:

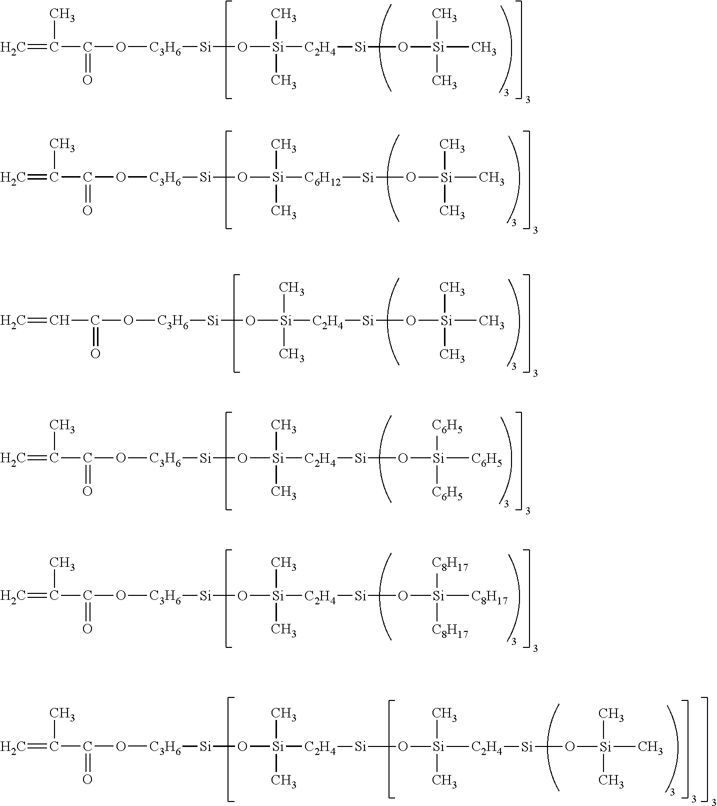

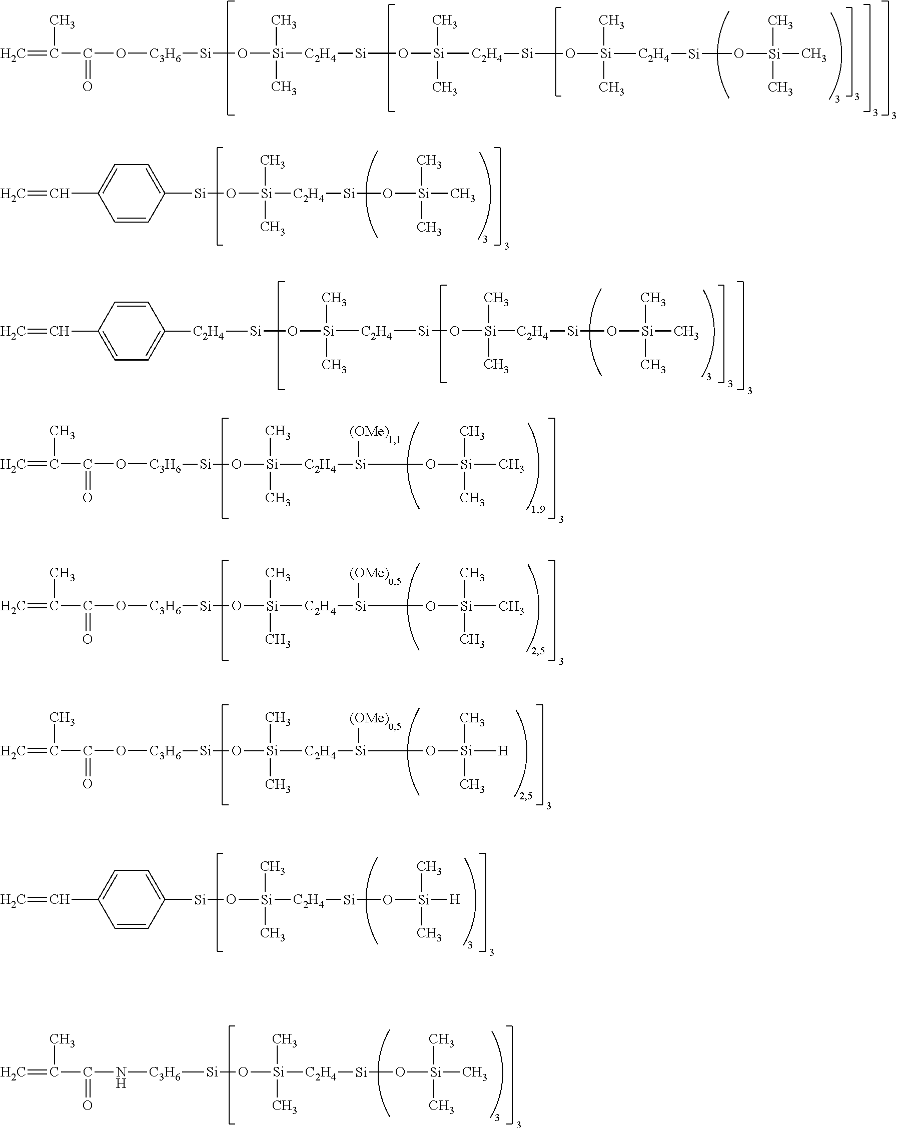

Thus, according to one embodiment, the carbosiloxane dendrimer of the composition according to the present invention is represented by the following formula:

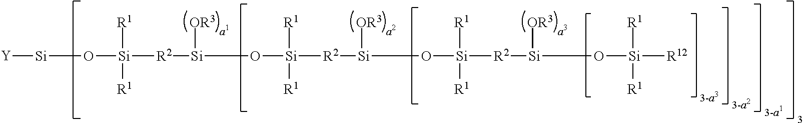

in which:
Y, R¹, R² and R³ are as defined in formulae (I) and (II) above;
a¹, a² and a³ correspond to the definition of a′ according to formula (II); and
R¹² is H, an aryl group containing from 5 to 10 carbon atoms or an alkyl group containing from 1 to 10 carbon atoms.

According to one embodiment, the carbosiloxane dendrimer of the composition according to the present invention is represented by one of the following formulae:

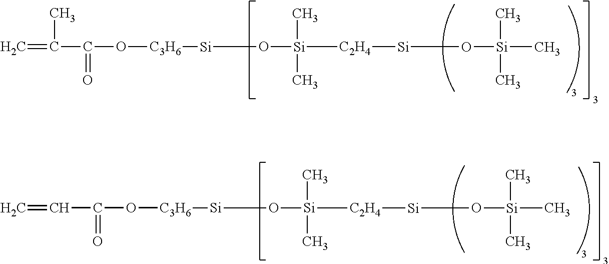

The vinyl polymer comprising the carbosiloxane dendrimer according to the invention may be manufactured according to the process for manufacturing a branched silalkylene siloxane described in Japanese patent application Hei 9-171 154.

For example, it may be produced by subjecting an organosilicon compound containing a hydrogen atom linked to a silicon atom, represented by the following general formula (IV):

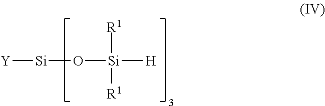

R¹ being as defined above in formula (I),

and an organosilicon compound containing an alkenyl group.

In the above formula, the organosilicon compound may be represented by 3-methacryloxypropyltris(dimethylsiloxy)silane, 3-acryloxypropyltris(dimethylsiloxy) silane and 4-vinylphenyltris(dimethylsiloxy)silane. The organosilicon compound that contains an alkenyl group may be represented by vinyltris(trimethylsiloxy)silane, vinyltris(dimethylphenylsiloxy)silane, and 5-hexenyltris(trimethylsiloxy)silane.

The hydrosilylation reaction is performed in the presence of a chloroplatinic acid, a complex of vinylsiloxane and of platinum, or a similar transition metal catalyst.

A vinyl polymer bearing at least one carbosiloxane dendrimer-based unit may be chosen from polymers such that the carbosiloxane dendrimer-based unit is a carbosiloxane dendritic structure represented by formula (III):

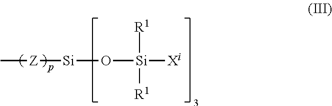

in which Z is a divalent organic group, “p” is 0 or 1, R¹ is as defined above in formula (IV) and Xⁱ is a silylalkyl group represented by formula (II) as defined above.

In a vinyl polymer bearing at least one carbosiloxane dendrimer-based unit, the polymerization ratio between the components (A) and (B), in terms of the weight ratio between (A) and (B), is within a range from 0/100 to 99.9/0.1, or even from 0.1/99.9 to 99.9/0.1 and preferably within a range from 1/99 to 99/1. A ratio between the components (A) and (B) of 0/100 means that the compound becomes a homopolymer of component (B).

A vinyl polymer bearing at least one carbosiloxane dendrimer-based unit may be obtained by copolymerization of the components (A) and (B), or by polymerization of the component (B) alone.

The polymerization may be a free-radical polymerization or an ionic polymerization, but free-radical polymerization is preferred.

The polymerization may be performed by bringing about a reaction between the components (A) and (B) in a solution for a period of from 3 to 20 hours in the presence of a radical initiator at a temperature of from 50° C. to 150° C.

A suitable solvent for this purpose is hexane, octane, decane, cyclohexane or a similar aliphatic hydrocarbon; benzene, toluene, xylene or a similar aromatic hydrocarbon; diethyl ether, dibutyl ether, tetrahydrofuran, dioxane or ethers; acetone, methyl ethyl ketone, methyl isobutyl ketone, diisobutyl ketone or similar ketones; methyl acetate, ethyl acetate, butyl acetate, isobutyl acetate or similar esters; methanol, ethanol, isopropanol, butanol or similar alcohols; octamethylcyclotetrasiloxane, decamethylcyclopentasiloxane, hexamethyldisiloxane, octamethyltrisiloxane or a similar organosiloxane oligomer.

A radical initiator may be any compound known in the art for standard free-radical polymerization reactions. The specific examples of such radical initiators are 2,2′-azobis(isobutyronitrile), 2,2′-azobis(2-methylbutyronitrile), 2,2′-azobis(2,4-dimethylvaleronitrile) or similar compounds of azobis type; benzoyl peroxide, lauroyl peroxide, tert-butyl peroxybenzoate, tert-butyl peroxy-2-ethylhexanoate or a similar organic peroxide. These radical initiators may be used alone or in a combination of two or more. The radical initiators may be used in an amount of from 0.1 to 5 parts by weight per 100 parts by weight of the components (A) and (B). A chain-transfer agent may be added. The chain-transfer agent may be 2-mercaptoethanol, butyl mercaptan, n-dodecyl mercaptan, 3-mercaptopropyltrimethoxysilane, a polydimethylsiloxane containing a mercaptopropyl group or a similar compound of mercapto type; methylene chloride, chloroform, carbon tetrachloride, butyl bromide, 3-chloropropyltrimethoxysilane or a similar halogenated compound.

In the manufacture of the polymer of vinyl type, after the polymerization, the unreacted residual vinyl monomer may be removed under conditions of heating under vacuum.

To facilitate the preparation of starting material for cosmetic products, the number-average molecular weight of the vinyl polymer bearing a carbosiloxane dendrimer may be chosen within the range between 3000 and 2 000 000 and preferably between 5000 and 800 000. It may be a liquid, a gum, a paste, a solid, a powder, or any other form. The preferred forms are solutions consisting of the dilution of a dispersion or of a powder in solvents.

The vinyl polymer may be a dispersion of a polymer of vinyl type bearing a carbosiloxane dendrimer structure in its side molecular chain, in a liquid such as a silicone oil, an organic oil, an alcohol or water.

The silicone oil may be a dimethylpolysiloxane having the two molecular ends capped with trimethylsiloxy groups, a copolymer of methylphenylsiloxane and of dimethylsiloxane having the two molecular ends capped with trimethylsiloxy groups, a copolymer of methyl-3,3,3-trifluoropropylsiloxane and of dimethylsiloxane having the two molecular ends capped with trimethylsiloxy groups, or similar unreactive linear silicone oils, and also hexamethylcyclotrisiloxane, octamethylcyclotetrasiloxane, decamethylcyclopentasiloxane, dodecamethylcyclohexasiloxane or a similar cyclic compound. In addition to the unreactive silicone oils, modified polysiloxanes containing functional groups such as silanol groups, amino groups and polyether groups on the ends or within the molecular side chains may be used.

The organic oils may be isododecane, liquid paraffin, isoparaffin, hexyl laurate, isopropyl myristate, myristyl myristate, cetyl myristate, 2-octyldodecyl myristate; isopropyl palmitate, 2-ethylhexyl palmitate, butyl stearate, decyl oleate, 2-octyldodecyl oleate, myristyl lactate, cetyl lactate, lanolin acetate, stearyl alcohol, cetostearyl alcohol, oleyl alcohol, avocado oil, almond oil, olive oil, cocoa oil, jojoba oil, gum oil, sunflower oil, soybean oil, camellia oil, squalane, castor oil, cottonseed oil, coconut oil, egg yolk oil, polypropylene glycol monooleate, neopentyl glycol 2-ethylhexanoate or a similar glycol ester oil; triglyceryl isostearate, the triglyceride of a fatty acid of coconut oil, or a similar oil of a polyhydric alcohol ester; polyoxyethylene lauryl ether, polyoxypropylene cetyl ether or a similar polyoxyalkylene ether.

The alcohol may be any type that is suitable for use in combination with a cosmetic product starting material. For example, it may be methanol, ethanol, butanol, isopropanol or similar lower alcohols.

A solution or a dispersion of the alcohol should have a viscosity within the range from 10 to 10⁹ mPa at 25° C. To improve the sensory use properties in a cosmetic product, the viscosity should be within the range from 100 to 5×10⁸ mPa·s.

The solutions and dispersions may be readily prepared by mixing a vinyl polymer bearing at least one carbosiloxane dendrimer-based unit with a silicone oil, an organic oil, an alcohol or water. The liquids may be present in the polymerization step. In this case, the unreacted residual vinyl monomer should be completely removed by heat treatment of the solution or dispersion under atmospheric pressure or reduced pressure.

In the case of a dispersion, the dispersity of the polymer of vinyl type may be improved by adding a surfactant.

Such an agent may be hexylbenzenesulfonic acid, octylbenzenesulfonic acid, decylbenzenesulfonic acid, dodecylbenzenesulfonic acid, cetylbenzenesulfonic acid, myristylbenzenesulfonic acid or anionic surfactants of the sodium salts of these acids; octyltrimethylammonium hydroxide, dodecyltrimethylammonium hydroxide, hexadecyltrimethylammonium hydroxide, octyldimethylbenzylammonium hydroxide, decyldimethylbenzylammonium hydroxide, dioctadecyldimethylammonium hydroxide, beef tallow-trimethylammonium hydroxide, coconut oil-trimethylammonium hydroxide, or a similar cationic surfactant; a polyoxyalkylene alkyl ether, a polyoxyalkylenealkylphenol, a polyoxyalkylene alkyl ester, the sorbitol ester of polyoxyalkylene, polyethylene glycol, polypropylene glycol, an ethylene oxide additive of diethylene glycol trimethylnonanol, and nonionic surfactants of polyester type, and also mixtures.

In the dispersion, a mean particle diameter of the polymer of vinyl type may be within a range of between 0.001 and 100 microns and preferably between 0.01 and 50 microns. The reason for this is that, outside the recommended range, a cosmetic product mixed with the emulsion will not have a nice enough feel on the skin or to the touch, or sufficient spreading properties or a pleasant feel.

A vinyl polymer contained in the dispersion or the solution may have a concentration within a range of between 0.1% and 95% by weight and preferably between 5% and 85% by weight. However, to facilitate the handling and the preparation of the mixture, the range should preferably be between 10% and 75% by weight.

A vinyl polymer that is suitable for use in the invention may also be one of the polymers described in the examples of patent application EP 0 963 751.

According to one preferred embodiment, a vinyl polymer grafted with a carbosiloxane dendrimer may be the product of polymerization of:

(A) from 0 to 99.9 parts by weight of one or more acrylate or methacrylate monomers; and

(B) from 100 to 0.1 parts by weight of an acrylate or methacrylate monomer of a tris[tri(trimethylsiloxy)silylethyldimethylsiloxy]silylpropyl carbosiloxane dendrimer.

The monomers (A1) and (B1) correspond respectively to specific monomers (A) and (B).

According to one embodiment, a vinyl polymer bearing at least one carbosiloxane dendrimer-based unit may comprise a tris[tri(trimethylsiloxy)silylethyldimethylsiloxy]silylpropyl carbosiloxane dendrimer-based unit corresponding to one of the formulae:

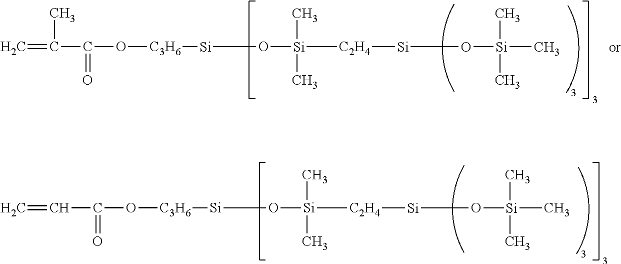

According to one preferred mode, a vinyl polymer bearing at least one carbosiloxane dendrimer-based unit used in the invention comprises at least one butyl acrylate monomer.

According to one embodiment, a vinyl polymer may also comprise at least one fluoro organic group.

Structures in which the polymerized vinyl units constitute the backbone and carbosiloxane dendritic structures and also fluoro organic groups are attached to side chains are particularly preferred.

The fluoro organic groups may be obtained by replacing with fluorine atoms all or some of the hydrogen atoms of methyl, ethyl, propyl, isopropyl, butyl, isobutyl, tert-butyl, pentyl, neopentyl, hexyl, cyclohexyl, heptyl, octyl, nonyl, decyl, undecyl, dodecyl, tridecyl, tetradecyl, hexadecyl and octadecyl groups and other alkyl groups of 1 to 20 carbon atoms, and also alkyloxyalkylene groups of 6 to 22 carbon atoms.

The groups represented by the formula —(CH₂)ₓ—(CF₂)_{y}—R¹³ are suggested as examples of fluoroalkyl groups obtained by substituting fluorine atoms for hydrogen atoms of alkyl groups. In the formula, the index “x” is 0, 1, 2 or 3, and “y” is an integer from 1 to 20. R¹³ is an atom or a group chosen from a hydrogen atom, a fluorine atom, —CH(CF₃)₂— or CF(CF₃)₂. Such fluorine-substituted alkyl groups are exemplified by linear or branched polyfluoroalkyl or perfluoroalkyl groups represented by the formulae shown below: —CF₃, —C₂F₅, -nC₃F₇, —CF(CF₃)₂, -nC₄F₉, CF₂CF(CF₃)₂, -nC₅F₁₁, -nC₆F₁₃, -nC₈F₁₇, CH₂CF₃, —(CH(CF₃)₂, CH₂CH(CF₃)₂—CH₂(CF₂)₂F, —CH₂(CF₂)₃F, —CH₂(CF₂)₄F, CH₂(CF₂)₆F, CH₂(CF₂)₈F, —CH₂CH₂CF₃, —CH₂CH₂(CF₂)₂F, —CH₂CH₂(CF₂)₃F, —CH₂CH₂(CF₂)₄F, —CH₂CH₂(CF₂)₆F, —CH₂CH₂(CF₂)₈F, —CH₂CH₂(CF₂)₁₀F, —CH₂CH₂(CF₂)₁₂F, CH₂CH₂(CF₂)₁₄F, —CH₂CH₂(CF₂)₁₆F, —CH₂CH₂CH₂CF₃, —CH₂CH₂CH₂(CF₂)₂F, —CH₂CH₂CH₂(CF₂)₂H, —CH₂(CF₂)₄H and —CH₂CH₂(CF₂)₃H.

The groups represented by —CH₂CH₂—(CF₂)ₘ—CFR¹⁴—[OCF₂CF(CF₃)]ₙ—OC₃F₇ are suggested as fluoroalkyloxyfluoroalkylene groups obtained by substituting fluorine atoms for hydrogen atoms of alkyloxyalkylene groups. In the formula, the index “m” is 0 or 1, “n” is 0, 1, 2, 3, 4 or 5, and R¹⁴ is a fluorine atom or CF₃. Such fluoroalkyloxyfluoroalkylene groups are exemplified by the perfluoroalkyloxyfluoroalkylene groups represented by the formulae shown below: —CH₂CH₂CF(CF₃)—[OCF₂CF(CF₃)]ₙ—OC₃F₇, —CH₂CH₂CF₂CF₂—[OCF₂CF(CF₃)]ₙ—OC₃F₇.

The number-average molecular weight of the vinyl polymer used in the present invention may be between 3000 and 2 000 000 and more preferably between 5000 and 800 000.

This type of fluorinated vinyl polymer may be obtained by addition:
of a vinyl monomer (M2) without a fluoro organic group,
on a vinyl monomer (M1) containing fluoro organic groups, and
a carbosiloxane dendrimer (B) as defined above, of general formula (I) as defined above,

by subjecting them to a copolymerization.

Thus, according to one embodiment, a composition of the invention may comprise a vinyl polymer bearing at least one carbosiloxane dendrimer-based unit and which results from the copolymerization of a vinyl monomer (M1) as defined above, optionally of a vinyl monomer (M2) as defined above, and of a carbosiloxane dendrimer (B) as defined above,

said vinyl polymer having a copolymerization ratio between the monomer (M1) and the monomer (M2) of 0.1 to 100:99.9 to 0% by weight, and a copolymerization ratio between the sum of the monomers (M1) and (M2) and the monomer (B) of 0.1 to 99.9:99.9 to 0.1% by weight.

The vinyl monomers (M1) containing fluoro organic groups in the molecule are preferably monomers represented by the general formula:

(CH²)═CR¹⁵COOR^{f}.

In this formula, R¹⁵ is a hydrogen atom or a methyl group and R^{f} is a fluoro organic group exemplified by the fluoroalkyl and fluoroalkyloxyfluoroalkylene groups described above. The compounds represented by the formulae presented below are suggested as specific examples of the component (M1). In the formulae presented below, “z” is an integer from 1 to 4.
CH₂═CCH₃COO—CF₃, CH₂═CCH₃COO—C₂F₅, CH₂═CCH3COO-nC₃F₇, CH₂═CCH₃COO—CF(CF₃)₂, CH₂═CCH₃COO-nC₄F₉, CH₂═CCH₃COO—CF(CF₃)₂, CH₂═CCH₃COO-nC₅F₁₁, CH₂═CCH₃COO-nC₆F₁₃, CH₂═CCH₃COO-nC₈F₁₇, CH₂═CCH₃COO—CH₂CF₃, CH₂═CCH₃COO—CH(CF₃)₂, CH₂═CCH₃COO—CH₂CH(CF₃)₂, CH₂═CCH₃COO—CH₂ (CF₂)₂F, CH₂═CCH₃COO—CH₂(CF₂)₂F, CH₂═CCH₃COO—CH₂(CF₂)₄F, CH₂═CCH₃COO—CH₂(CF₂)₆F, CH₂═CCH₃COO—CH₂(CF₂)₈F, CH₂═CCH₃COO—CH₂CH₂CF₃, CH₂—CCH₃COO—CH₂CH₂(CF₂)₂F, CH₂—CCH₃COO—CH₂CH₂(CF₂)₃F, CH₂—CCH₃COO—CH₂CH₂(CF₂)₄F, CH₂—CCH₃COO—CH₂CH₂(CF₂)₆F, CH₂═CCH₃COO—CH₂CH₂(CF₂)₈F, CH₂═CCH₃COO—CH₂CH₂(CF₂)₁₀F, CH₂—CCH₃COO—CH₂CH₂(CF₂)₁₂F, CH₂—CCH₃COO—CH₂CH₂(CF₂)₁₄F, CH₂—CCH₃COO—CH₂—CH₂—(CF₂)₁₆F, CH₂—CCH₃COO—CH₂CH₂CH₂CF₃, CH₂═CCH₃COO—CH₂CH₂CH₂(CF₂)₂F, CH₂═CCH₃ COO—CH₂CH₂CH₂(CF₂)₂H, CH₂═CCH₃COO—CH₂ (CF₂)₄H, CH₂═CCH₃COO—(CF₂)₃H, CH₂═CCH₃COO—CH₂CH₂CF(CF₃)—[OCF₂—CF(CF₃)]z-OC₃F₇, CH₂═CCH₃COO—CH₂CH₂CF₂CF₂—[OCF₂—CF(CF₃)]z-OC₃F₇, CH₂═CHCOO—CF₃, CH2=CHCOO—C₂F₅, CH₂═CHCOO-nC₃F₇, CH₂═CHCOO—CF(CF₃)₂, CH₂═CHCOO-nC₄F₉, CH₂═CHCOO—CF₂CF(CF₃)₂, CH₂═CHCOO-nC₅F₁₁, CH₂═CHCOO-nC₆F₁₃, CH₂═CHCOO-nC₈F₁₇, CH₂═CHCOO—CH₂CF₃, CH₂═CHCOO—CH(CF₃)₂, CH₂═CHCOO—CH₂CH(CF₃)₂, CH₂═CHCOO—CH₂(CF₂)₂F, CH₂═CHCOO—CH₂(CF₂)₃F, CH₂═CHCOO—CH₂(CF₂)₄F, CH₂═CHCOO—CH₂(CF₂)₆F, CH₂═CHCOO—CH₂(CF₂)₈F, CH₂═CHCOO—CH₂CH₂CF₃, CH₂═CHCOO—CH₂CH₂ (CF₂)₂F, CH₂═CHCOO—CH₂CH₂(CF₂)₃F, CH₂═CHCOO—CH₂CH₂(CF₂)₄F, CH₂═CHCOO—CH₂CH₂(CF₂)₆F, CH₂═CHCOO—CH₂CH₂(CF₂)₈F, CH₂═HCOO—CH₂CH₂(CF₂)₁₀F, CH₂—CHCOO—CH₂CH₂—(CF₂)₁₂F, CH₂═CHCOO—CH₂CH₂(CF₂)₁₄F, CH₂═CHCOO—CH₂CH₂(CF₂)₁₆F, CH₂═CHCOO—CH₂CH₂CH₂CF₃, CH₂═CHCOO—CH₂CH₂CH₂(CF₂)₂F, CH₂═CHCOO—CH₂CH₂CH₂(CF)₂H, CH₂═CHCOO—CH₂(CF₂)₄H, CH₂═CHCOO—CH₂CH₂(CF₂)₃H, CH₂═CHCOO—CH₂CH₂CF(CF₃)—, [OCF₂—CF(CF₃)]_{z}—OC₃F₇, CH₂═CHCOO—CH₂CH₂CF₂CF₂(CF₃)—[OCF₂—CF(CF₃)]₂-0C₃F₇.

Among these, the vinyl polymers represented by the formulae presented below are preferable:
CH₂═CHCOO—CH₂CH₂(CF₂)₆F, CH₂═CHCOO—CH₂CH₂(CF₂)₈F, CH₂═CCH₃COO—CH₂CH₂(CF₂)₆F, CH₂═CCH₃COO—CH₂CH₂(CF₂)₈F, CH₂═CHCOO—CH₂CF₃, CH₂═CCH₃COO—CH₂CF₃.

The vinyl polymers represented by the formulae presented below are particularly preferable:

CH₂═CHCOO—CH₂CF₃,CH₂═CCHCOO—CH₂CF₃.

The vinyl monomers (M2) not containing any organofluorine groups in the molecule may be any monomers containing radical-polymerizable vinyl groups which are exemplified, for example, by methyl acrylate, methyl methacrylate, ethyl acrylate, ethyl methacrylate, n-propyl acrylate, n-propyl methacrylate, isopropyl acrylate, isopropyl methacrylate, and other lower alkyl acrylates or methacrylates; glycidyl acrylate, glycidyl methacrylate; n-butyl acrylate, n-butyl methacrylate, isobutyl acrylate, isobutyl methacrylate, tert-butyl acrylate, tert-butyl methacrylate, n-hexyl acrylate, n-hexyl methacrylate, cyclohexyl acrylate, cyclohexyl methacrylate, 2-ethylhexyl acrylate, 2-ethylhexyl methacrylate, octyl acrylate, octyl methacrylate, lauryl acrylate, lauryl methacrylate, stearyl acrylate, stearyl methacrylate, and other higher acrylates and methacrylates; vinyl acetate, vinyl propionate and other lower fatty acid vinyl esters; vinyl butyrate, vinyl caproate, vinyl 2-ethylhexanoate, vinyl laurate, vinyl stearate, and other higher fatty acid esters; styrene, vinyltoluene, benzyl acrylate, benzyl methacrylate, phenoxyethyl acrylate, phenoxyethyl methacrylate, vinylpyrrolidone, and other vinyl aromatic monomers; dimethylaminoethyl acrylate, dimethylaminoethyl methacrylate, diethylaminoethyl acrylate, diethylaminoethyl methacrylate, and other aminovinyl monomers, acrylamide, methacrylamide, N-methylolacrylamide, N-methylolmethacrylamide, N-methoxymethylacrylamide, N-methoxymethylmethacrylamide, isobutoxymethoxyacrylamide, isobutoxymethoxymethacrylamide, N,N-dimethylacrylamide, N,N-dimethylmethacrylamide, and other vinylamide monomers; hydroxyethyl acrylate, hydroxyethyl methacrylate, acrylic acid hydroxypropyl alcohol, methacrylic acid hydroxypropyl alcohol, and other hydroxyvinyl monomers; acrylic acid, methacrylic acid, itaconic acid, crotonic acid, fumaric acid, maleic acid, and other vinylcarboxylic acid monomers; tetrahydrofurfuryl acrylate, tetrahydrofurfuryl methacrylate, butoxyethyl acrylate, butoxyethyl methacrylate, ethoxydiethylene glycol acrylate, ethoxydiethylene glycol methacrylate, polyethylene glycol acrylate, polyethylene glycol methacrylate, polypropylene glycol monoacrylate, polypropylene glycol monomethacrylate, hydroxybutyl vinyl ether, cetyl vinyl ether, 2-ethylhexyl vinyl ether, and other vinyl monomers containing an ether bond; acryloxypropyltrimethoxysilane, methacryloxypropyltrimethoxysilane, polydimethylsiloxanes containing acryl or methacryl groups at one of the ends, polydimethylsiloxanes containing alkenylaryl groups at one of the ends and other silicone compounds containing unsaturated groups; butadiene; vinyl chloride; vinylidene chloride, acrylonitrile, methacrylonitrile; dibutyl fumarate; maleic anhydride; dodecylsuccinic anhydride; acryl glycidyl ether, methacryl glycidyl ether, 3,4-epoxycyclohexylmethyl acrylate, 3,4-epoxycyclohexylmethyl methacrylate, alkali metal salts, ammonium salts and organic amine salts of acrylic acid, of methacrylic acid, of itaconic acid, of crotonic acid, of fumaric acid, of maleic acid and of other radical-polymerizable unsaturated carboxylic acids, radical-polymerizable unsaturated monomers containing sulfonic acid groups, such as styrene sulfonic acid and also the alkali metal salts thereof, the ammonium salts thereof and the organic amine salts thereof; the quaternary ammonium salts derived from acrylic acid or methacrylic acid, such as 2-hydroxy-3-methacryloxypropyltrimethylammonium chloride, methacrylic acid esters of a tertiary amine alcohol, such as the diethylamine ester of methacrylic acid and quaternary ammonium salts thereof.

In addition, it is also possible to use as vinyl monomers (M2) the polyfunctional vinyl monomers illustrated, for example, by trimethylolpropane triacrylate, trimethylolpropane trimethacrylate, pentaerythrityl triacrylate, pentaerythrityl trimethacrylate, ethylene glycol diacrylate, ethylene glycol dimethacrylate, tetraethylene glycol diacrylate, tetraethylene glycol dimethacrylate, polyethylene glycol diacrylate, polyethylene glycol dimethacrylate, 1,4-butanediol diacrylate, 1,4-butanediol dimethacrylate, 1,6-hexanediol diacrylate, 1,6-hexanediol dimethacrylate, neopentyl glycol diacrylate, neopentyl glycol dimethacrylate, trimethylolpropanetrioxyethyl acrylate, trimethylolpropanetrioxyethyl methacrylate, tris(2-hydroxyethyl)isocyanurate diacrylate, tris(2-hydroxyethyl)isocyanurate dimethacrylate, tris(2-hydroxyethyl)isocyanurate triacrylate, tris(2-hydroxyethyl)isocyanurate trimethacrylate, polydimethyl siloxane in which the two ends of the molecular chain are blocked with alkenylaryl groups, and other silicone compounds containing unsaturated groups.

As regards the ratio mentioned above in which (M1) and (M2) are copolymerized, the weight ratio between (M1) and (M2) is preferably within the range 1:99 to 100:0.

Y can be chosen, for example, from organic groups containing acrylic or methacrylic groups, organic groups containing an alkenylaryl group, or alkenyl groups containing from 2 to 10 carbon atoms.

The organic groups containing acrylic or methacrylic groups and the alkenylaryl groups are as defined above.

Among the compounds (B), mention may, for example, be made of the following compounds:

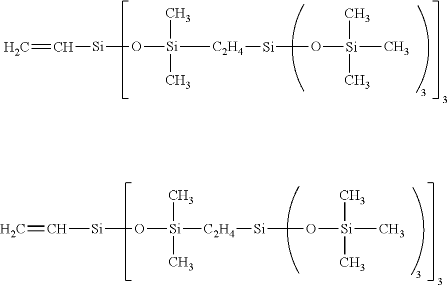

The carbosiloxane dendrimers (B) may be prepared using the process for preparing siloxane/silalkylene branched copolymers described in document EP 1 055 674.

For example, they may be prepared by subjecting organic alkenyl silicone compounds and silicone compounds comprising hydrogen atoms bonded to the silicon, represented by formula (IV) as defined above, to a hydrosilylation reaction.

The copolymerization ratio (by weight) between the monomer (B) and the monomers (M1) and (M2) is preferably within the range from 1:99 to 99:1 and even more preferably within the range from 5:95 to 95:5.

Amino groups may be introduced into the side chains of the vinyl polymer using, included in the component (M2), vinyl monomers containing amino groups, such as dimethylaminoethyl acrylate, dimethylaminoethyl methacrylate, diethylaminoethyl acrylate and diethylaminoethyl methacrylate, followed by performing a modification with potassium acetate monochloride, ammonium acetate monochloride, the aminomethylpropanol salt of monochloroacetic acid, the triethanolamine salt of monobromoacetic acid, sodium monochloropropionate, and other alkali metal salts of halogenated fatty acids; otherwise, carboxylic acid groups may be introduced into the side chains of the vinyl polymer using, included in the component (M2), vinyl monomers containing carboxylic acids, such as acrylic acid, methacrylic acid, itaconic acid, crotonic acid, fumaric acid and maleic acid, and the like, followed by neutralizing the product with triethylamine, diethylamine, triethanolamine and other amines.

A fluorinated vinyl polymer may be one of the polymers described in the examples of patent application WO 03/045 337.

According to one preferred embodiment, a vinyl polymer grafted in the sense of the present invention may be conveyed in an oil or a mixture of oils, which is/are preferably volatile, chosen in particular from silicone oils and hydrocarbon-based oils, and mixtures thereof.

According to one particular embodiment, a silicone oil that is suitable for use in the invention may be cyclopentasiloxane.

According to another particular embodiment, a hydrocarbon-based oil that is suitable for use in the invention may be isododecane.

Vinyl polymers grafted with at least one carbosiloxane dendrimer-based unit that may be particularly suitable for use in the present invention are the polymers sold under the names TIB 4-100, TIB 4-101, TIB 4-120, TIB 4-130, TIB 4-200, FA 4002 ID (TIB 4-202), TIB 4-220 and FA 4001 CM (TIB 4-230) by the company Dow Corning.

According to one embodiment, the composition according to the present invention comprises the vinyl polymer bearing at least one carbosiloxane dendrimer-based unit in an active material content ranging from 3% to 15% by weight relative to the total weight of the composition, in particular ranging from 4% to 10% by weight and more particularly ranging from 5% to 8% by weight of active material of vinyl polymer bearing at least one carbosiloxane dendrimer-based unit.

Additional Fillers

A composition in accordance with the invention may also advantageously comprise at least one additional filler, of organic or mineral nature, which makes it possible especially to give it additional properties of coverage, and improved matt effect and/or stability with regard to exudation and properties of non-migration after application.

In particular, the composition of the invention comprises at least one additional organic filler, and optionally also at least one mineral filler. Preferably, the organic filler/mineral filler weight ratio is greater than 1.

The term “filler” should be understood as meaning colourless or white, solid particles of any shape, which are in an insoluble and dispersed form in the medium of the composition. Being of mineral or organic nature, they make it possible to impart body or rigidity to the composition, and/or softness, a matt effect and uniformity to the makeup result.

The fillers used in the compositions according to the present invention may be in lamellar, globular or spherical form, in the form of fibres or in any other form intermediate between these defined forms.

Preferably, the composition of the invention at least comprises spherical fillers.

The fillers according to the invention may or may not be surface-coated, and in particular they may be surface-treated with silicones, amino acids, fluoro derivatives or any other substance that promotes the dispersion and compatibility of the filler in the composition.

The term “organic fillers” should be understood as meaning colourless or white, lamellar or non-lamellar organic particles. Mention may be made especially of polyamide powders (Nylon® or Orgasol® from Arkema), acrylic polymer powders, especially powders of polymethyl methacrylate, of polymethyl methacrylate/ethylene glycol dimethacrylate, of polyallyl methacrylate/ethylene glycol dimethacrylate, of ethylene glycol dimethacrylate/lauryl methacrylate copolymer, cellulose powders, poly-β-alanine powders and polyethylene powders, powders of tetrafluoroethylene polymers (Teflon®), lauroyllysine, starch, hollow polymer microspheres of acrylic acid copolymers (Polytrap® from the company Dow Corning) and silicone resin microbeads (for example Tospearls® from Toshiba), elastomeric polyorganosiloxane particles, obtained especially by polymerization of organopolysiloxane containing at least two hydrogen atoms each bonded to a silicon atom and of an organopolysiloxane comprising at least two ethylenically unsaturated groups (especially two vinyl groups) in the presence of a platinum catalyst, or alternatively metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate.

The term “mineral fillers” should be understood as meaning colourless or white, lamellar or non-lamellar mineral particles. The mineral fillers may be of any form, especially such as potato-shaped, lamellar, platelet-shaped, spherical or oblong. They may be of any crystallographic form (for example lamellar, cubic, hexagonal, orthorhombic, etc.). Mention may be made especially of talc, mica, silica, kaolin, boron nitride, precipitated calcium carbonate, magnesium carbonate, magnesium hydrogen carbonate, hydroxyapatite, hollow silica microspheres (Silica Beads® from Maprecos) or glass or ceramic microcapsules, clay, quartz, natural diamond powder, or a mixture thereof.

Matt-Effect or Sebum-Absorbing Fillers

According to a preferred mode, a person skilled in the art is interested in the context of the invention in additional matt-effect fillers.

Thus, according to a particular mode, the composition of the invention comprises, as additional filler, at least one matt-effect or sebum-absorbing filler (filler with a capacity for absorbing and/or adsorbing an oil or a liquid fatty substance, for instance sebum (from the skin)).

This oil-absorbing filler may also advantageously have a BET specific surface area of greater than or equal to 300 m²/g, preferably greater than 500 m²/g and preferentially greater than 600 m²/g, and especially less than 1500 m²/g.

The BET specific surface area is determined according to the BET (Brunauer-Emmett-Teller) method described in the Journal of the American Chemical Society, vol. 60, page 309, February 1938, which corresponds to International Standard ISO 5794/1 (appendix D). The BET specific surface area corresponds to the total specific surface area (thus including micropores) of the powder.

The matt-effect filler may thus have an oil uptake of greater than or equal to 1 ml/g, especially ranging from 1.5 ml/g to 20 ml/g, or even ranging from 1.5 ml/g to 15 ml/g. This oil uptake, which corresponds to the amount of oil absorbed and/or adsorbed by the filler, may be characterized by measuring the wet point according to the method described below.

Method for Measuring the Oil Uptake of a Filler:

The oil uptake of a powder is measured according to the method for determining the oil uptake of a powder described in standard NF T 30-022. It corresponds to the amount of oil adsorbed onto the available surface of the filler, by measuring the wet point.

An amount m (in grams) of powder of between about 0.5 g and 5 g (the amount depends on the density of the powder) is placed on a glass plate and isononyl isononanoate is then added dropwise.

After addition of 4 to 5 drops of isononyl isononanoate, the isononyl isononanoate is incorporated into the filler using a spatula, and addition of the isononyl isononanoate is continued until a conglomerate of isononyl isononanoate and powder has formed. At this point, the isononyl isononanoate is added one drop at a time and the mixture is then triturated with the spatula. The addition of isononyl isononanoate is stopped when a firm, smooth paste is obtained. This paste must be able to be spread over the glass plate without cracks or the formation of lumps. The volume Vs (expressed in ml) of isononyl isononanoate used is then noted.

The oil uptake corresponds to the ratio Vs/m.

The matt-effect filler according to the invention may be of organic or mineral nature.

It may be chosen more particularly from silicas, silica silylates, polyamide powders (in particular Nylon-6 or Nylon-12), acrylic polymer powders, especially polymethyl methacrylate, polymethyl methacrylate/ethylene glycol dimethacrylate, polyallyl methacrylate/ethylene glycol dimethacrylate or ethylene glycol dimethacrylate/lauryl methacrylate copolymer powders; perlites; magnesium carbonate, and mixtures thereof.

According to a particularly preferred mode, the composition of the invention comprises at least one matt-effect organic filler, chosen in particular from polyamide powders such as Nylon® or Orgasol® from Arkema.

In this respect, mention may be made of Nylon 6, Nylon 6-6, Nylon 12 or Nylon 6-12 particles, such as those sold by the company Atofina under the generic name Orgasol.

Preferably, the porous organic particles of Nylon type are chosen from Nylon-12 particles.

The composition of the invention may thus comprise from 2% to 15% by weight of organic fillers, preferably with a matt effect, relative to the total weight of the composition, in particular from 3% to 10% and even more particularly from 5% to 8% by weight relative to the total weight of the composition.

According to an advantageous mode, the composition comprises at least one matt-effect organic filler and also at least one mineral filler, preferably a silica.

A composition according to the invention thus advantageously comprises at least one organic filler, chosen in particular from polyamide powders, optionally also combined with a mineral filler such as a silica.

The presence of silica in a composition according to the invention is especially advantageous since it further contributes towards improving the cosmetic properties of the said composition.

A silica that is suitable for use in the invention is a hydrophilic or hydrophobic silica chosen from precipitated silicas and fumed silicas, and mixtures thereof.

A silica that is suitable for use in the invention may be of spherical or non-spherical, porous or non-porous form.

Preferably, a silica that is suitable for use in the invention is spherical and porous.

Mention may be made, as matt-effect silica powder, of:
the porous silica microspheres sold under the name Silica Beads SB-700 by the company Miyoshi or the name Sunsphere® H51 or Sunsphere® H33 by the company Asahi Glass; and
the polydimethylsiloxane-coated amorphous silica microspheres sold under the name SA Sunsphere® H33 or SA Sunsphere® H53 by the company Asahi Glass.

Mention may be made, as matt-effect silica powder, of:
porous silica microspheres, especially those sold under the names Sunsphere® H53 and Sunsphere® H33 by the company Asahi Glass; MSS-500-3H by the company Kobo,
polydimethylsiloxane-coated amorphous silica microspheres, especially those sold under the name SA Sunsphere® H33,
silica silylate powders, especially those sold under the name Dow Corning VM-2270 Aerogel Fine Particles by the company Dow Corning, and
amorphous hollow silica particles, especially those sold under the name Silica Shells by the company Kobo.

A composition of the invention may comprise from 0.5% to 15% by weight of mineral filler, preferably with a matt effect, such as a silica, relative to the total weight of the composition, in particular from 2% to 10% and even more particularly from 3% to 7% by weight relative to the total weight of the composition.

The total content of fillers in the composition, including the expanded polymer particles and the abovementioned additional fillers, may vary within a wide range depending on the nature of the filler and also depending on the desired product and/or the desired effects; this amount is to be adapted by a person skilled in the art.

Thus, the total content of filler(s), especially when the composition comprises additional fillers that are different from the expanded polymer particles, will range from 3% to 20% by weight relative to the total weight of the composition, preferably from 8% to 15% by weight or even from 10% to 15% by weight relative to the total weight of the said composition.

Thus, according to a particularly advantageous embodiment, a composition according to the invention uses a “vinyl polymer(s) bearing at least one carbosiloxane dendrimer derivative/total fillers” weight ratio, especially when the composition comprises additional fillers that are different from the expanded polymer particles, of less than 1, preferably less than 0.6 and better still less than 0.5.

Physiologically Acceptable Medium

Besides the compounds indicated previously, a composition according to the invention comprises a physiologically acceptable medium.

The term “physiologically acceptable medium” is intended to denote a medium that is particularly suitable for applying a composition of the invention to keratin materials, in particular the skin, the lips or the eyebrows.

The physiologically acceptable medium is generally adapted to the nature of the support onto which the composition has to be applied, and also to the appearance under which the composition has to be packaged.

As indicated previously, a composition according to the invention is an inverse emulsion (W/O).

Aqueous Phase

The composition according to the invention comprises an aqueous phase.

The aqueous phase comprises water. A water that is suitable for use in the invention may be a floral water such as cornflower water and/or a mineral water such as Vittel water, Lucas water or La Roche Posay water and/or a spring water.

The aqueous phase may also comprise water-miscible organic solvents (at room temperature: 25° C.), for instance monoalcohols containing from 2 to 6 carbon atoms, such as ethanol or isopropanol; polyols especially containing from 2 to 20 carbon atoms, preferably containing from 2 to 10 carbon atoms and preferentially containing from 2 to 6 carbon atoms, such as glycerol, propylene glycol, butylene glycol, pentylene glycol, hexylene glycol, dipropylene glycol or diethylene glycol; glycol ethers (especially containing from 3 to 16 carbon atoms) such as mono-, di- or tripropylene glycol (C₁-C₄)alkyl ethers, mono-, di- or triethylene glycol (C₁-C₄)alkyl ethers, and mixtures thereof.

The aqueous phase may also comprise stabilizers, for example sodium chloride, magnesium dichloride or magnesium sulfate.

The aqueous phase may also comprise any water-soluble or water-dispersible compound that is compatible with an aqueous phase, such as gelling agents, film-forming polymers, thickeners or surfactants, and mixtures thereof.

In particular, a composition of the invention may comprise an aqueous phase in a content ranging from 1% to 80% by weight, especially from 5% to 50% and more particularly from 10% to 45% by weight relative to the total weight of the composition.

Liquid Fatty Phase

A cosmetic composition in accordance with the present invention may comprise at least one liquid fatty phase and especially at least one oil as mentioned below.

The term “oil” means any fatty substance that is in liquid form at room temperature (20-25° C.) and at atmospheric pressure.

A composition of the invention may comprise a liquid fatty phase in a content ranging from 1% to 90%, in particular from 5% to 80%, in particular from 10% to 70% and more particularly from 20% to 50% by weight relative to the total weight of the composition.

The oily phase that is suitable for preparing the cosmetic compositions according to the invention may comprise, besides the abovementioned oils in relation with the vinyl polymer grafted with a carbosiloxane dendrimer, hydrocarbon-based oils, silicone oils, fluoro oils or non-fluoro oils, or mixtures thereof.

Preferably, the composition of the invention comprises at least one silicone oil.

The oils may be volatile or non-volatile.

They may be of animal, plant, mineral or synthetic origin.

For the purposes of the present invention, the term “volatile oil” means an oil (or non-aqueous medium) that is capable of evaporating on contact with the skin in less than one hour, at room temperature and at atmospheric pressure. The volatile oil is a volatile cosmetic oil, which is liquid at room temperature, especially having a non-zero vapour pressure, at room temperature and atmospheric pressure, in particular having a vapour pressure ranging from 0.13 Pa to 40 000 Pa (10⁻³ to 300 mmHg), preferably ranging from 1.3 Pa to 13 000 Pa (0.01 to 100 mmHg) and preferably ranging from 1.3 Pa to 1300 Pa (0.01 to 10 mmHg).

For the purposes of the present invention, the term “non-volatile oil” means an oil with a vapour pressure of less than 0.13 Pa.

For the purposes of the present invention, the term “silicone oil” is understood to mean an oil comprising at least one silicon atom, and in particular at least one Si—O group.

The term “fluoro oil” is intended to mean an oil comprising at least one fluorine atom.

The term “hydrocarbon-based oil” is intended to mean an oil comprising mainly hydrogen and carbon atoms.

The oils may optionally comprise oxygen, nitrogen, sulfur and/or phosphorus atoms, for example in the form of hydroxyl or acid radicals.

Volatile Oils

The volatile oils may be chosen from hydrocarbon-based oils containing from 8 to 16 carbon atoms, and especially C₈-C₁₆ branched alkanes (also known as isoparaffins), for instance isododecane (also known as 2,2,4,4,6-pentamethylheptane), isodecane and isohexadecane, for instance the oils sold under the trade names Isopar® or Permethyl®.

Volatile oils that may also be used include volatile silicones, for instance volatile linear or cyclic silicone oils, especially those with a viscosity 8 centistokes (cSt) (8×10⁻⁶ m²/s), and especially containing from 2 to 10 silicon atoms and in particular from 2 to 7 silicon atoms, these silicones optionally comprising alkyl or alkoxy groups containing from 1 to 10 carbon atoms. As volatile silicone oils that may be used in the invention, mention may be made especially of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile fluoro oils such as nonafluoromethoxybutane or perfluoromethylcyclopentane, and mixtures thereof, may also be used.

According to one embodiment, a composition of the invention may comprise from 1% to 80% by weight, or even from 5% to 70% by weight, or even from 10% to 60% by weight and especially from 15% to 50% by weight of volatile oils relative to the total weight of the composition, this content taking into account the volatile oil conveying the vinyl polymer bearing a carbosiloxane dendrimer unit.

Non-Volatile Oils

The non-volatile oils may be chosen especially from non-volatile hydrocarbon-based, fluoro and/or silicone oils.

Non-volatile hydrocarbon-based oils that may especially be mentioned include:

hydrocarbon-based oils of animal origin, such as perhydrosqualene,

hydrocarbon-based oils of plant origin, such as phytostearyl esters, such as phytostearyl oleate, phytostearyl isostearate and lauroyl/octyldodecyl/phytostearyl glutamate (Ajinomoto, Eldew PS203), triglycerides formed from fatty acid esters of glycerol, in particular in which the fatty acids may have chain lengths ranging from C₄ to C₃₆ and especially from C₁₈ to C₃₆, these oils possibly being linear or branched, and saturated or unsaturated; these oils may especially be heptanoic or octanoic triglycerides, shea oil, alfalfa oil, poppy oil, winter squash oil, millet oil, barley oil, quinoa oil, rye oil, candlenut oil, passionflower oil, shea butter, aloe vera oil, sweet almond oil, peach stone oil, groundnut oil, argan oil, avocado oil, baobab oil, borage oil, broccoli oil, calendula oil, camelina oil, canola oil, carrot oil, safflower oil, flax oil, rapeseed oil, cotton oil, coconut oil, marrow seed oil, wheatgerm oil, jojoba oil, lily oil, macadamia oil, corn oil, meadowfoam oil, St John's Wort oil, monoi oil, hazelnut oil, apricot kernel oil, walnut oil, olive oil, evening primrose oil, palm oil, blackcurrant pip oil, kiwi seed oil, grapeseed oil, pistachio oil, winter squash oil, pumpkin oil, quinoa oil, musk rose oil, sesame oil, soybean oil, sunflower oil, castor oil and watermelon oil, and mixtures thereof, or alternatively caprylic/capric acid triglycerides, such as those sold by the company Stéarineries Dubois or those sold under the names Miglyol 810®, 812® and 818® by the company Dynamit Nobel;

linear or branched hydrocarbons, of mineral or synthetic origin, such as liquid paraffins and derivatives thereof, petroleum jelly, polydecenes, polybutenes, hydrogenated polyisobutene such as Parleam, or squalane;

synthetic ethers containing from 10 to 40 carbon atoms;

synthetic esters, for instance oils of formula R₁COOR₂, in which R₁ represents a linear or branched fatty acid residue containing from 1 to 40 carbon atoms, and R₂ represents a hydrocarbon-based chain that is especially branched, containing from 1 to 40 carbon atoms provided that R₁+R₂≧10. The esters may be chosen especially from fatty alcohol and fatty acid esters, for instance cetostearyl octanoate, isopropyl alcohol esters such as isopropyl myristate or isopropyl palmitate, ethyl palmitate, 2-ethylhexyl palmitate, isopropyl stearate or isostearate, isostearyl isostearate, octyl stearate, hydroxylated esters, for instance isostearyl lactate, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and 2-ethylhexyl palmitate, alkyl benzoate, polyethylene glycol diheptanoate, propylene glycol diethyl-2-hexanoate, and mixtures thereof, C₁₂-C₁₅ alkyl benzoates, hexyl laurate, neopentanoic acid esters, for instance isodecyl neopentanoate, isotridecyl neopentanoate, isostearyl neopentanoate and octyldodecyl neopentanoate, isononanoic acid esters, for instance isononyl isononanoate, isotridecyl isononanoate and octyl isononanoate, hydroxylated esters, for instance isostearyl lactate and diisostearyl malate;

polyol esters and pentaerythritol esters, for instance dipentaerythrityl tetrahydroxystearate/tetraisostearate,

esters of diol dimers and of diacid dimers, such as Lusplan DD-DA5® and Lusplan DD-DA7® sold by the company Nippon Fine Chemical and described in patent application US 2004-175 338,

copolymers of a diol dimer and of a diacid dimer and esters thereof, such as dilinoleyl diol dimer/dilinoleic dimer copolymers and esters thereof, for instance Plandool-G,

copolymers of polyols and of diacid dimers, and esters thereof, such as Hailuscent ISDA or the dilinoleic acid/butanediol copolymer,

fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol,

C₁₂-C₂₂ higher fatty acids, such as oleic acid, linoleic acid and linolenic acid, and mixtures thereof, and

dialkyl carbonates, the two alkyl chains possibly being identical or different, such as dicaprylyl carbonate sold under the name Cetiol CC® by Cognis; and

oils of high molar mass, in particular with a molar mass ranging from about 400 to about 10 000 g/mol, in particular from about 650 to about 10 000 g/mol, in particular from about 750 to about 7500 g/mol and more particularly ranging from about 1000 to about 5000 g/mol. As oils of high molar mass that may be used in the present invention, mention may especially be made of oils chosen from:
lipophilic polymers,
linear fatty acid esters with a total carbon number ranging from 35 to 70,
hydroxylated esters,
aromatic esters,
C₂₄-C₂₈ branched fatty acid or fatty alcohol esters,
silicone oils,
oils of plant origin,
and mixtures thereof;
optionally partially hydrocarbon-based and/or silicone fluoro oils, for instance fluorosilicone oils, fluoropolyethers and fluorosilicones as described in document EP-A-847 752;
silicone oils, for instance linear or cyclic non-volatile polydimethylsiloxanes (PDMS); polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates, and
mixtures thereof.

According to a particular embodiment, the fatty phase of the composition according to the invention may contain only volatile compounds.

Advantageously, and as indicated previously, a composition according to the invention may comprise at least one silicone oil or a mixture of silicone oils. According to a particular mode, the composition of the invention comprises at least one volatile silicone oil, preferably dodecamethylpentasiloxane.

Advantageously, a cosmetic composition in accordance with the present invention may also comprise at least one hydrocarbon-based volatile oil, chosen from isododecane and isohexadecane, and mixtures thereof.

Lipophilic Structuring Agent

A composition according to the invention, in particular when it is an eyeshadow composition, may also comprise at least one agent for structuring the liquid fatty phase, chosen from a wax and a pasty compound, and mixtures thereof.

In particular, a wax that is suitable for use in the invention may be chosen especially from waxes of animal, plant, mineral or synthetic origin, and mixtures thereof.

As examples of waxes that may be used according to the invention, mention may be made of:
waxes of animal origin, such as beeswax, spermaceti, lanolin wax and lanolin derivatives, plant waxes such as carnauba wax, candelilla wax, ouricury wax, Japan wax, cocoa butter, cork fibre wax or sugarcane wax,
mineral waxes, for example paraffin wax, petroleum jelly wax, lignite wax, microcrystalline waxes or ozokerites,
synthetic waxes, including polyethylene waxes and the waxes obtained by Fisher-Tropsch synthesis,
silicone waxes, in particular substituted linear polysiloxanes; examples that may be mentioned include polyether silicone waxes, alkyl or alkoxy dimethicones containing from 16 to 45 carbon atoms, alkyl methicones such as the C₃₀-C₄₅ alkyl methicone sold under the trade name AMS C 30 by Dow Corning,
hydrogenated oils that are solid at 25° C., such as hydrogenated castor oil, hydrogenated jojoba oil, hydrogenated palm oil, hydrogenated tallow or hydrogenated coconut oil, and fatty esters that are solid at 25° C., for instance the C₂₀-C₄₀ alkyl stearate sold under the trade name Kester Wax K82H by the company Koster Keunen,
and/or mixtures thereof.

Preferably, use will be made of polyethylene waxes, microcrystalline waxes, carnauba waxes, hydrogenated jojoba oil, candelilla waxes, beeswaxes and ozokerites, and/or mixtures thereof.

The structuring agent(s) may be present in a composition of the invention in a content ranging from 0.1% to 30% by weight of agents and more preferably from 0.5% to 20% by weight, relative to the total weight of the composition.

Thickeners

Depending on the fluidity of the composition that it is desired to obtain, it is possible to incorporate one or more thickeners or gelling agents into a composition of the invention.

A thickener or gelling agent that is suitable for use in the invention may be hydrophilic, i.e. soluble or dispersible in water.

Hydrophilic gelling agents that may be mentioned in particular include water-soluble or water-dispersible thickening polymers. These polymers may be chosen especially from: modified or unmodified carboxyvinyl polymers, such as the products sold under the name Carbopol (CTFA name: Carbomer) by the company Goodrich; polyacrylates and polymethacrylates such as the products sold under the names Lubrajel and Norgel by the company Guardian or under the name Hispagel by the company Hispano Chimica; polyacrylamides; optionally crosslinked and/or neutralized 2-acrylamido-2-methylpropanesulfonic acid polymers and copolymers, for instance the poly(2-acrylamido-2-methylpropanesulfonic acid) sold by the company Clariant under the name Hostacerin AMPS (CTFA name: ammonium polyacryldimethyltauramide); crosslinked anionic copolymers of acrylamide and of AMPS, which are in the form of a W/O emulsion, such as those sold under the name Sepigel 305 (CTFA name: Polyacrylamide/C13-14 Isoparaffin/Laureth-7) and under the name Simulgel 600 (CTFA name Acrylamide/Sodium acryloyldimethyltaurate copolymer/Isohexadecane/Polysorbate 80) by the company SEPPIC; polysaccharide biopolymers, for instance xanthan gum, guar gum, carob gum, acacia gum, scleroglucans, chitin and chitosan derivatives, carrageenans, gellans, alginates, celluloses such as microcrystalline cellulose, carboxymethyl cellulose, hydroxymethyl cellulose and hydroxypropyl cellulose; and mixtures thereof.

A thickener or gelling agent that is suitable for use in the invention may be lipophilic. It may be mineral or organic.

As lipophilic thickeners, mention may be made, for example, of modified clays such as modified magnesium silicate (Bentone gel VS38 from Rheox), modified hectorites such as hectorite modified with a C₁₀ to C₂₂ fatty acid ammonium chloride, for instance hectorite modified with distearyldimethylammonium chloride such as, for example, that sold under the name Bentone 38V® by the company Elementis or that sold under the name “Bentone 38 CE” by the company Rheox or that sold under the name Bentone Gel V5 5V by the company Elementis.

Among the lipophilic gelling agents that may be used in a cosmetic composition of the invention, mention may also be made of fatty acid esters of dextrin, such as dextrin palmitates, especially such as those sold under the names Rheopearl TL® or Rheopearl KL® by the company Chiba Flour, hydrogenated plant oils, such as hydrogenated castor oil, fatty alcohols, in particular of C₈ to C₂₆ and more particularly of C₁₂ to C₂₂, for instance myristyl alcohol, cetyl alcohol, stearyl alcohol or behenyl alcohol.

According to one embodiment, a composition of the invention may comprise thickeners in an active material content from 0.01% to 40% by weight, especially from 0.1% to 20% by weight and in particular from 0.3% to 15% by weight relative to the total weight of the composition.

Dyestuffs

A composition according to the invention may also comprise at least one dyestuff.

A cosmetic composition in accordance with the invention may advantageously incorporate at least one dyestuff chosen from organic or mineral dyestuffs, especially such as the pigments or nacres conventionally used in cosmetic compositions, liposoluble or water-soluble dyes, materials with a specific optical effect, and mixtures thereof.

The term “pigments” should be understood as meaning white or coloured, mineral or organic particles that are insoluble in an aqueous solution and that are intended to colour and/or opacify the resulting film.

The pigments may be present in a proportion of from 0.1% to 40% by weight, especially from 1% to 30% by weight and in particular from 5% to 15% by weight relative to the total weight of the cosmetic composition.

As mineral pigments that may be used in the invention, mention may be made of titanium oxides, zirconium oxides or cerium oxides, and also zinc oxides, iron oxides or chromium oxides, ferric blue, manganese violet, ultramarine blue and chromium hydrate.

The pigment may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

The dyestuff may also comprise a pigment having a structure which may be, for example, of the type such as silica microspheres containing iron oxide. An example of a pigment having this structure is the product sold by the company Miyoshi under the reference PC Ball PC-LL-100 P, this pigment consisting of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPP) described in documents EP-A-542 669, EP-A-787 730, EP-A-787 731 and WO-A-96/08537.

The term “nacres” should be understood as meaning iridescent or non-iridescent coloured particles of any form, especially produced by certain molluscs in their shell or alternatively synthesized, which have a colour effect via optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the commercially available nacres that may be mentioned are the nacres Timica, Flamenco and Duochrome (on mica base) sold by the company Engelhard, the Timiron nacres sold by the company Merck, the Prestige nacres on mica base sold by the company Eckart and the Sunshine nacres on synthetic mica base sold by the company Sun Chemical.

The nacres may more particularly have a yellow, pink, red, bronze, orange, brown, gold and/or coppery colour or tint.

As illustrations of nacres that may be used in the context of the present invention, mention may especially be made of gold-coloured nacres sold especially by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company Merck under the names Bronze fine (17384) (Colorona) and Bronze (17353) (Colorona) and by the company Engelhard under the name Super bronze (Cloisonne); the orange nacres sold especially by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold especially by the company Engelhard under the names Nu-antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red-tinted nacres with a golden tint sold especially by the company Engelhard under the name Sunstone G012 (Gemtone); the pink nacres sold especially by the company Engelhard under the name Tan opale G005 (Gemtone); the black nacres with a golden tint sold especially by the company Engelhard under the name Nu antique bronze 240 AB (Timica); the blue nacres sold especially by the company Merck under the name Matte blue (17433) (Microna); the white nacres with a silvery tint sold especially by the company Merck under the name Xirona Silver; and the golden-green pinkish-orange nacres sold especially by the company Merck under the name Indian summer (Xirona), and mixtures thereof.

The cosmetic composition according to the invention may also comprise water-soluble or liposoluble dyes. The liposoluble dyes are, for example, Sudan red, DC Red 17, DC Green 6, β-carotene, soybean oil, Sudan brown, DC Yellow 11, DC Violet 2, DC Orange 5 and quinoline yellow. The water-soluble dyes are, for example, beetroot juice and caramel.

Additives

A cosmetic composition according to the invention may also comprise any additive usually used in the field concerned, chosen, for example, from gums, anionic, cationic, amphoteric or nonionic surfactants, silicone surfactants, resins, dispersants, semi-crystalline polymers, antioxidants, essential oils, preserving agents, fragrances, neutralizers, antiseptics, UV stabilizers, cosmetic or dermatological active agents, such as vitamins, moisturizers, emollients or collagen-protecting agents, desquamating agents, agents for improving the barrier function, depigmenting agents, antiacne agents, calmatives and anti-inflammatory agents, and mixtures thereof.

It is a matter of routine operations for a person skilled in the art to adjust the nature and amount of the additives present in the compositions in accordance with the invention such that the cosmetic properties thereof, especially the desired matt effect thereof and its persistence over time, and the desired stability properties thereof, are not thereby affected.

A composition according to the invention may especially be in the form of a composition for making up and/or caring for the skin, the lips or the eyebrows, in particular a foundation or a makeup base.

Throughout the description, including the claims, the expression “comprising a” should be understood as being synonymous with “comprising at least one”, unless otherwise specified.

The terms “between . . . and . . . ” and “ranging from . . . to . . . ” should be understood as being inclusive of the limits, unless otherwise specified.

The examples and figures that follow are presented as non-limiting illustrations of the invention.

### EXAMPLES

### Example 1

### Influence of the Nature of the Polymer on the Persistence of the Matt Effect

General Formula of Example 1 (Fluid Foundation)

| | | Example 1A | Example 1B | Example 1C |
|---|---|---|---|---|
| Phase | Compounds | (Invention) | (comparative) | (comparative) |
| A1 | Bis-PEG/PPG-14/14 dimethicone (and) | 1.00 | 1.00 | 1.00 |
| | dimethicone sold under the reference | | | |
| | Abil EM 97S by the company Evonik | | | |
| | Goldschmidt | | | |
| | Isododecane | 0.00 | 12.5 | 5.50 |
| | Isohexadecane | 6.50 | 6.50 | 6.50 |
| | PEG-10 dimethicone sold under the | 2.50 | 2.50 | 2.50 |
| | reference KF-6017 by the company | | | |
| | Shin-Etsu | | | |
| | Acrylates/ | 12.50 (i.e. | 0.00 | 0.00 |
| | polytrimethylsiloxy methacrylate | 5% active | | |
| | copolymer sold under the reference | material) | | |
| | Dow Corning FA 4002 ID Silicone | | | |
| | Acrylate by the company Dow Corning | | | |
| | (40% active material) | | | |
| | polypropylsilsesquioxane (and) | 0.00 | 0.00 | 6.9 (i.e. |
| | isododecane Tpropyl resin sold under | | | 5% active |
| | the reference Dow Corning 680 ID | | | material) |
| | Fluid by the company Dow Corning | | | |
| | (72.5-77.5% active material) | | | |
| | Qs dimethicone sold under the | qs 100 | qs 100 | qs 100 |
| | reference DM-Fluid 2 CS by the | | | |
| | company Shin-Etsu | | | |
| A2 | Disteardimonium hectorite sold under | 0.35 | 0.35 | 0.35 |
| | the reference Bentone 38 VCG by the | | | |
| | company Elementis | | | |
| A3 | Iron and titanium oxides | 13.00 | 13.00 | 13.00 |
| A4 | Nylon-12 sold under the reference | 7.00 | 7.00 | 7.00 |
| | Orgasol 2002 EXD NAT COS by the | | | |
| | company Arkema | | | |
| | Silica sold under the reference | 3.00 | 3.00 | 3.00 |
| | Sunsphere H51 AGC by the company | | | |
| | Si-Tech | | | |
| | Acrylate copolymer sold under the | 0.50 | 0.50 | 0.50 |
| | reference Expancel 551 DE 40 D42 by | | | |
| | the company Akzo Nobel | | | |
| B1 | Water | 32.00 | 32.00 | 32.00 |
| B2 | Glycol | 4.00 | 4.00 | 4.00 |
| | Phenoxyethanol | 1.20 | 1.20 | 1.20 |

Operating Protocol:

The constituents of phase B2 are weighed out in a 500 ml beaker.

In parallel, bring B1 to boil and then add B1 to B2.

Place under magnetic stirring to cool.

Weigh out A1 in a 1 L beaker and stir with a Moritz blender.

Weigh out A2 in a capsule and then add A2 to A1.

Weigh out A3+50% of A1 in a capsule. Mill three times in a three-roll mill and then add the rest of A1+A2.

Weigh out the fillers of A4 separately and introduce them one by one, allowing a minimum of five minutes of homogenization between each addition.

The emulsion is made at room temperature: the mixture B1 and B2 is poured into the fatty phase while gradually increasing the stirring speed (Moritz blender). Stirring is continued for 10 minutes.

Measurement of the Matt Effect and the Persistence of the Matt Effect

The matt effect and the persistence of the matt effect are evaluated on a panel of seven women (A to G) according to the protocol detailed previously in the description.

The reference Example 1A was evaluated as being matt at T0 and as persisting very well on the face after 3 hours. The level of mattness after 3 hours is judged to be equal to that at T0.

The panel was requested to judge the performance qualities in terms of the matt effect and the persistence of the matt effect of compositions B and C on half the face, relative to the reference Example 1A.

The differences are noted with 1 or 2+ if the immediate matt effect or the persistence of the matt effect at 3 hours is better and 1 or 2− in the contrary case.

The results obtained with compositions 1A to C are given in Table 1 below.

TABLE 1

| | | | |
|---|---|---|---|
| | Example 1A | Example 1B | Example 1C |
| Matt | (Invention) | (comparative) | (comparative) |

| effect | T0 | T3 h | T0 | T3 h | T0 | T3 h |
|---|---|---|---|---|---|---|
| A | 0 | 0 | = | −− | = | −− |
| B | 0 | 0 | = | −− | = | − |
| C | 0 | 0 | = | − | = | −− |
| D | 0 | 0 | = | −− | = | −− |
| E | 0 | 0 | = | = | = | − |
| F | 0 | 0 | = | − | = | − |
| G | 0 | 0 | = | −− | = | −− |

The fluid foundation 1A (according to the invention) shows improved performance qualities as regards the persistence of the matt effect when compared with foundations 1B and 1C (comparative). Specifically, the vinyl polymer bearing at least one carbosiloxane dendrimer-based unit (1A) is superior to the other polymers (1B and 1C) with regard to the persistence of the matt effect.

What is more, composition 1A is pleasant and easy to apply, while at the same time maintaining satisfactory comfort on application i.e. not causing any sensation of tautness or mask effect throughout the day and/or not inducing any greasy or tacky sensation during its application.

### Example 2

### Influence of the Nature of the Filler on the Persistence of the Matt Effect

General Formula of Example 2 (Fluid Foundation)

| | | Example 2A | Example 2B | Example 2C |
|---|---|---|---|---|
| Phase | Compounds | (Invention) | (comparative) | (comparative) |
| A1 | Bis-PEG/PPG-14/14 dimethicone | 1.00 | 1.00 | 1.00 |
| | (and) dimethicone sold under the | | | |
| | reference Abil EM 97S by the | | | |
| | company Evonik Goldschmidt | | | |
| | Isohexadecane | 6.50 | 6.50 | 6.50 |
| | PEG-10 dimethicone sold under the | 2.50 | 2.50 | 2.50 |
| | reference KF-6017 by the company | | | |
| | Shin-Etsu | | | |
| | Acrylates/ | 12.50 (i.e. | 12.50 (i.e. | 12.50 (i.e. |
| | polytrimethylsiloxy methacrylate | 5% active | 5% active | 5% active |
| | copolymer sold under the reference | material) | material) | material) |
| | Dow Corning FA 4002 ID Silicone | | | |
| | Acrylate by the company Dow | | | |
| | Corning (40% active material) | | | |
| | Qs dimethicone sold under the | qs 100 | qs 100 | qs 100 |
| | reference DM-Fluid 2 CS by the | | | |
| | company Shin-Etsu | | | |
| A2 | Disteardimonium hectorite sold under | 0.35 | 0.35 | 0.35 |
| | the reference Bentone 38 VCG by the | | | |
| | company Elementis | | | |
| A3 | Iron and titanium oxides | 13.00 | 13.00 | 13.00 |
| A5 | Nylon-12 sold under the reference | 7.00 | 7.25 | 7.10 |
| | Orgasol 2002 EXD NAT COS by the | | | |
| | company Arkema | | | |
| | Silica sold under the reference | 3.00 | 3.25 | 3.10 |
| | Sunsphere H51 by the company | | | |
| | Arkema | | | |
| | Acrylate copolymer sold under the | 0.50 | 0.00 | 0.30 |
| | reference Expancel 551 DE 40 D42 by | | | |
| | the company Akzo Nobel | | | |
| B1 | Water | 32.00 | 32.00 | 32.00 |
| B2 | Glycol | 4.00 | 4.00 | 4.00 |
| | Phenoxyethanol | 1.20 | 1.20 | 1.20 |

Example 2A is identical to Example 1A and prepared in the same manner.

Measurement of the Matt Effect and the Persistence of the Matt Effect

This measurement is as described in Example 1 above.

The results obtained with compositions 2A to C are given in Table 2 below.

TABLE 2

| | | | |
|---|---|---|---|
| | Example 2A | Example 2B | Example 2C |
| Matt | (Invention) | (comparative) | (comparative) |

| effect | T0 | T3h | T0 | T3h | T0 | T3h |
|---|---|---|---|---|---|---|
| A | 0 | 0 | = | − | = | − |
| B | 0 | 0 | = | − | = | − |
| C | 0 | 0 | = | −− | = | − |
| D | 0 | 0 | = | −− | = | − |
| E | 0 | 0 | = | − | = | = |
| F | 0 | 0 | = | = | = | − |
| G | 0 | 0 | = | −− | = | − |

The fluid foundation 2A (according to the invention) shows improved performance qualities as regards the persistence of the matt effect when compared with foundations 2B and 2C (comparative). Specifically, the presence of the Expancel particles in a specific content at least equal to 0.5% by weight (2A) is necessary to obtain properties in terms of a matt effect and persistence of the matt effect over time.

What is more, since the composition 2A is in fact identical to the composition 1A of Example 1, the same comments regarding the application properties and the comfort may be repeated.

In the light of the foregoing, it is seen that it is the combination Expancel (at at least 0.5% by weight)+vinyl polymer bearing at least one carbosiloxane dendrimer-based unit which makes it possible to obtain an improved cosmetic effect, namely substantial persistence of the matt effect.

The shades of the composition of Example 1A described above may be adapted as a function of the application, on facial skin or the eyebrows, by varying the content of iron oxides (especially black iron oxide) and of titanium oxides. A composition of the invention as described in Example 1 and with a pigment content adjusted to the desired shade may thus constitute an eyebrow makeup product having an immediate matt effect and a persistent matt effect over time.

### Example 3

### Eyeshadow Composition

| Phase | Compounds | Example |
|---|---|---|
| A1 | Bis-PEG/PPG-14/14 dimethicone (and) | 1.00 |
| | dimethicone sold under the reference Abil | |
| | EM 97S by the company Evonik Goldschmidt | |
| | Isododecane | 2.5 |
| | PEG-10 dimethicone sold under the | 2.50 |
| | reference KF-6017 by the company Shin- | |
| | Etsu | |
| | Acrylates/ | 12.50 (i.e. |
| | polytrimethylsiloxy methacrylate copolymer | 5% active |
| | sold under the reference Dow Corning FA | material) |
| | 4002 ID Silicone Acrylate by the company | |
| | Dow Corning (40% active material) | |
| A1 | Dimethicone sold under the reference DM- | qs 100 |
| | Fluid 2 CS by the company Shin-Etsu | |
| A2 | Disteardimonium hectorite sold under the | 0.35 |
| | reference Bentone 38 VCG by the company | |
| | Elementis | |
| A3 | Iron and titanium oxides | 13.00 |
| A4 | Nylon-12 sold under the reference Orgasol | 7.00 |
| | 2002 EXD NAT COS by the company | |
| | Arkema | |
| | Silica sold under the reference Sunsphere | 3.00 |
| | H51 by the company Arkema | |
| | Acrylate copolymer sold under the reference | 0.50 |
| | Expancel 551 DE 40 D42 by the company | |
| | Akzo Nobel | |
| B1 | Water | 32.00 |
| B2 | Glycol | 4.00 |
| | Phenoxyethanol | 1.20 |
| C | Ozokerite | 6.50 |
| | Stearyl alcohol | 10 |
| | Isohexadecane | 6 |

Operating Protocol:

The constituents of phase B2 are weighed out in a 500 ml beaker.

In parallel, bring B1 to boil and then add B1 to B2.

Place under magnetic stirring to cool.

Weigh out A1 in a 1 L beaker and stir with a Moritz blender.

Weigh out A2 in a capsule and then add A2 to A1.

Weigh out A3+50% of A1 in a capsule. Mill three times in a three-roll mill and then add the rest of A1+A2.

Weigh out the fillers of A4 separately and introduce them one by one, allowing a minimum of five minutes of homogenization between each addition.

The emulsion is made at room temperature: the mixture B1 and B2 is poured into the fatty phase while gradually increasing the stirring speed (Moritz blender). Stirring is continued for 10 minutes.

In parallel, melt phase C on a water bath at 60° C.

Cool to 40° C. and introduce into the emulsion with stirring using a Rayneri blender. Continue stirring for 10 minutes.

The final product is a solid composition, which, when applied to the eyelids, gives a fine makeup result with improved immediate matt-effect properties and improved persistence of the matt effect over time.

### Example 4

### Care or Makeup Base

| | | Example |
|---|---|---|
| Phase | Compounds | (Invention) |
| A1 | Bis-PEG/PPG-14/14 dimethicone (and) | 1.15 |
| | dimethicone sold under the reference Abil | |
| | EM 97S by the company Evonik Goldschmidt | |
| | Isohexadecane | 7.45 |
| | PEG-10 dimethicone sold under the | 2.85 |
| | reference KF-6017 by the company Shin- | |
| | Etsu | |
| | Acrylates/polytrimethylsiloxy methacrylate | 14.35 (i.e. |
| | copolymer sold under the reference Dow | 5.74% active |
| | Corning FA 4002 ID Silicone Acrylate by the | material) |
| | company Dow Corning (40% active material) | |
| | Dimethicone sold under the reference DM- | qs 100 |
| | Fluid 2 CS by the company Shin-Etsu | |
| A2 | Disteardimonium hectorite sold under the | 0.40 |
| | reference Bentone 38 VCG by the company | |
| | Elementis | |
| A3 | Iron and titanium oxides | 0 |
| A4 | Nylon-12 sold under the reference Orgasol | 8.05 |
| | 2002 EXD NAT COS by the company | |
| | Arkema | |
| | Silica sold under the reference Sunsphere | 3.45 |
| | H51 by the company Arkema | |
| | Acrylate copolymer sold under the reference | 0.55 |
| | Expancel 551 DE 40 D42 by the company | |
| | Akzo Nobel | |
| B1 | Water | 36.8 |
| B2 | Glycol | 4.60 |
| | Phenoxyethanol | 1.40 |

This composition is prepared according to the protocol described in Example 1.

When applied to facial skin before a makeup product (e.g.: a foundation), it makes it possible to improve the matt result of the makeup and the persistence of the matt effect over time.

1-17. (canceled)

18. A cosmetic composition for making up and/or caring for keratin materials, in the form of a water-in-oil emulsion comprising, in a physiologically acceptable medium, at least one vinyl polymer bearing at least one carbosiloxane dendrimer-based unit, and at least 0.5% by weight of expanded polymer particles relative to the total weight of the composition.

19. The composition according to claim 18, wherein the expanded polymer particles are chosen from particles of thermoplastic materials chosen from polymers or copolymers of acrylonitrile, of vinylidene chloride, of vinyl chloride and/or of an acrylic or styrene monomer.

20. The composition according to claim 18, wherein the expanded polymer particles are chosen from hollow particles of an expanded copolymer of vinylidene chloride and acrylonitrile, or of vinylidene chloride, acrylonitrile and methyl methacrylate.

21. The composition according to claim 18, wherein the said composition comprises from 0.5% to 5% by weight of expanded polymer particles relative to the total weight of the composition.

22. The composition according to claim 18, wherein the said composition comprises from 0.5% to 2.5% by weight of expanded polymer particles relative to the total weight of the composition.

23. The composition according to claim 18, wherein the said composition comprises from 0.5% to 1.5% by weight of expanded polymer particles relative to the total weight of the composition.

24. The composition according to claim 18, wherein the vinyl polymer bearing at least one carbosiloxane dendrimer-based unit has a molecular side chain containing a carbosiloxane dendrimer structure and is derived from the polymerization of:
(A) from 0 to 99.9 parts by weight of a vinyl monomer; and
(B) from 100 to 0.1 parts by weight of a carbosiloxane dendrimer of formula (I) below: 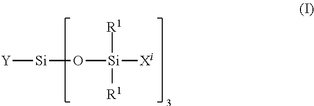
in which:
R¹ represents an aryl group containing from 5 to 10 carbon atoms or an alkyl group containing from 1 to 10 carbon atoms;
Xⁱ represents a silylalkyl group which, when i=1, is represented by formula (II): 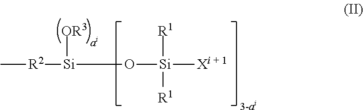
in which:
R¹ is as defined above in formula (I),
R² represents an alkylene radical containing from 2 to 10 carbon atoms,
R³ represents an alkyl group containing from 1 to 10 carbon atoms,
Xⁱ⁺¹ is chosen from: a hydrogen atom, an alkyl group containing from 1 to 10 carbon atoms, an aryl group containing from 5 to 10 carbon atoms and a silylalkyl group defined above of formula (II) with i=i+1,
i is an integer from 1 to 10 which represents the generation of said silylalkyl group, and
aⁱ is an integer from 0 to 3;
Y represents a radical-polymerizable organic group chosen from:
organic groups containing a methacrylic group or an acrylic group, said organic groups being represented by the formulae: 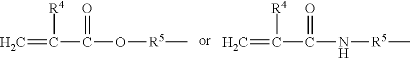
in which:
R⁴ represents a hydrogen atom or an alkyl group containing from 1 to 10 carbon atoms; and
R⁵ represents an alkylene group containing from 1 to 10 carbon atoms; and
organic groups containing a styryl group of formula: 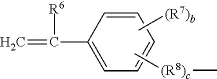
in which:
R⁶ represents a hydrogen atom or an alkyl group containing from 1 to 10 carbon atoms;
R⁷ represents an alkyl group containing from 1 to 10 carbon atoms;
R⁸ represents an alkylene group containing from 1 to 10 carbon atoms;
b is an integer from 0 to 4; and
c is 0 or 1, such that, if c is 0, —(R⁸)_{c}— represents a bond.

25. The composition according to claim 18, wherein the carbosiloxane dendrimer is represented by the following formula: 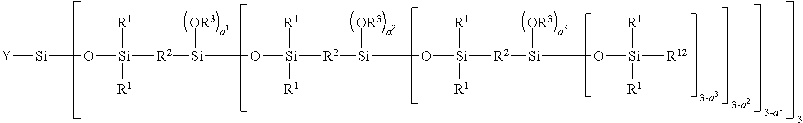
in which:
R¹ represents an aryl group containing from 5 to 10 carbon atoms or an alkyl group containing from 1 to 10 carbon atoms,
R² represents an alkylene radical containing from 2 to 10 carbon atoms,
R³ represents an alkyl group containing from 1 to 10 carbon atoms,
a¹, a² and a³ are independently an integer from 0 to 3;
Y represents a radical-polymerizable organic group chosen from:
organic groups containing a methacrylic group or an acrylic group, said organic groups being represented by the formulae: 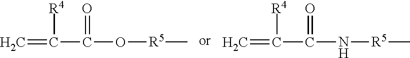
in which:
R⁴ represents a hydrogen atom or an alkyl group containing from 1 to 10 carbon atoms; and
R⁵ represents an alkylene group containing from 1 to 10 carbon atoms; and
organic groups containing a styryl group of formula: 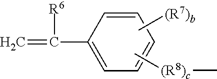
in which:
R⁶ represents a hydrogen atom or an alkyl group containing from 1 to 10 carbon atoms;
R⁷ represents an alkyl group containing from 1 to 10 carbon atoms;
R⁸ represents an alkylene group containing from 1 to 10 carbon atoms;
b is an integer from 0 to 4; and
c is 0 or 1, such that, if c is 0, —(R⁸)_{c}— represents a bond; and
R¹² is H, an aryl group containing from 5 to 10 carbon atoms or an alkyl group containing from 1 to 10 carbon atoms.

26. The composition according to claim 18, wherein the carbosiloxane dendrimer is represented by one of the following formulae:

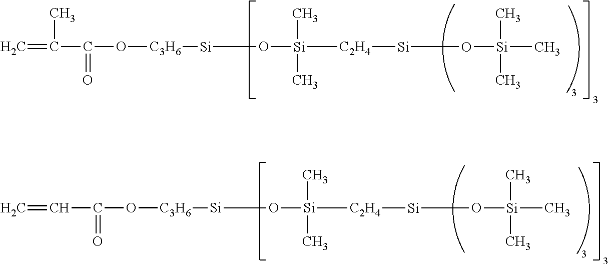

27. The composition according to claim 18, wherein the said vinyl polymer bearing at least one carbosiloxane dendrimer-based unit is present in an active material content ranging from 3% to 15% by weight relative to the total weight of the composition.

28. The composition according to claim 18, wherein the said vinyl polymer bearing at least one carbosiloxane dendrimer-based unit is present in an active material content ranging from 4% to 10% by weight relative to the total weight of the composition.

29. The composition according to claim 18, wherein the said vinyl polymer bearing at least one carbosiloxane dendrimer-based unit is present in an active material content ranging from 5% to 8% by weight relative to the total weight of the composition.

30. The composition according to claim 18, comprising a vinyl polymer(s) bearing at least one carbosiloxane dendrimer derivative/expanded polymer particles weight ratio of greater than or equal to 6.

31. The composition according to claim 18, comprising a vinyl polymer(s) bearing at least one carbosiloxane dendrimer derivative/expanded polymer particles weight ratio of greater than or equal to 8.

32. The composition according to claim 18, comprising a vinyl polymer(s) bearing at least one carbosiloxane dendrimer derivative/expanded polymer particles weight ratio of greater than or equal to 10.

33. The composition according to claim 18, wherein it further comprises at least one additional filler.

34. The composition according to claim 33, wherein the at least one additional filler is chosen from matt-effect fillers or sebum-absorbing fillers.

35. The composition according to claim 33, wherein the additional filler is an organic filler.

36. The composition according to claim 35, wherein the organic filler is chosen from polyamide powders, optionally also combined with a mineral filler.

37. The composition according to claim 18, wherein the total content of filler(s), especially when the composition comprises additional fillers that are different from the expanded polymer particles, ranges from 3% to 20% by weight relative to the total weight of the composition.

38. The composition according to claim 18, wherein the total content of filler(s), especially when the composition comprises additional fillers that are different from the expanded polymer particles, ranges from 8% to 15% by weight relative to the total weight of the composition.

39. The composition according to claim 18, wherein the total content of filler(s), especially when the composition comprises additional fillers that are different from the expanded polymer particles, ranges from 10% to 15% by weight relative to the total weight of the composition.

40. The composition according to claim 18, comprising a “vinyl polymer(s) bearing at least one carbosiloxane dendrimer derivative/total fillers” weight ratio, especially when the composition comprises additional fillers that are different from the expanded polymer particles, of less than 1.

41. The composition according to claim 18, comprising a “vinyl polymer(s) bearing at least one carbosiloxane dendrimer derivative/total fillers” weight ratio, especially when the composition comprises additional fillers that are different from the expanded polymer particles, of less than 0.6.

42. The composition according to claim 18, comprising a “vinyl polymer(s) bearing at least one carbosiloxane dendrimer derivative/total fillers” weight ratio, especially when the composition comprises additional fillers that are different from the expanded polymer particles, of less than 0.5.

43. The composition according to claim 18, further comprising at least one dyestuff.

44. The composition according to claim 18, the said composition being a care or makeup base, or a makeup product for keratin materials.

45. The composition according to claim 18, the said composition being a foundation, a hot-cast foundation product, a lipstick, a body makeup product, a concealer product, an eyeshadow or an eyebrow product.

46. A non-therapeutic process for making up and/or caring for keratin materials, comprising at least the step of applying to the said keratin material at least one coat of a composition according to claim 18.
